(19) United States
(12) Patent Application Publication
(10) Pub. No.: US 2018/0286507 A1
(43) Pub. Date: Oct. 4, 2018

(54) DIABETES MANAGEMENT SYSTEMS, METHODS AND APPARATUS FOR USER REMINDERS, PATTERN RECOGNITION, AND INTERFACES

(71) Applicant: Ascensia Diabetes Care Holdings AG, Basel (CH)

(72) Inventors: Jennifer L. Gass, Tarrytown, NY (US); Raymond L. Yao, Ossining, NY (US); Robert W. Morin, Boca Raton, FL (US); Lauren N. Bock, Ossining, NY (US); Vladislav Milenkovic, Merion Station, PA (US); Jeffery S. Reynolds, New Fairfield, CT (US); Eugene Prais, West Milford, NJ (US)

(21) Appl. No.: 15/936,370

(22) Filed: Mar. 26, 2018

Related U.S. Application Data
(60) Provisional application No. 62/478,023, filed on Mar. 28, 2017.

(51) Int. Cl. G16H 20/17; G16H 50/20; G16H 40/63; G06F 3/0484; G06F 3/0485; G06F 3/0482

(52) U.S. Cl. CPC G16H 20/17; G16H 50/20; G06F 3/0482; G06F 3/04847; G06F 3/0485; G16H 40/63

(57) ABSTRACT

Systems, methods, and apparatus for diabetes management include a portable diabetes management system (DMS) device. The DMS device includes a processor, a data storage device, a touchscreen display, and wireless communications facilities. An interactive display screen configured to be displayed on the touchscreen display lists a selectable subset of a plurality of different detected patterns related to blood glucose measurement data received by the DMS device. The patterns are detected based on a plurality of algorithms executable on the processor. A subset of detected patterns is determined based upon a frequency with which the patterns are detected and a priority is assigned to the detected patterns. Numerous other aspects are provided.

### CROSS REFERENCE TO RELATED APPLICATION

This claims the benefit of U.S. Provisional Application No. 62/478,023, filed Mar. 28, 2017, the disclosure of which is hereby incorporated by reference herein for all purposes.

### FIELD

The present invention relates to diabetes management systems and more specifically to such systems, methods, and apparatus for user reminders, pattern recognition, and interfaces.

### BACKGROUND

Diabetes mellitus is a serious, life-long disorder that is, as yet, incurable. Each year in the U.S. alone, about 2 million people are diagnosed with diabetes, the 7^{th} leading causes of death in the United States. In 2012, 86 million Americans age 20 and older had pre-diabetes; this is up from 79 million in 2010. In 1993, there were approximately eight million diagnosed cases of diabetes mellitus in the United States and the number has grown to about 21 million currently diagnosed cases. In addition, there are at least 8 mill ion undiagnosed cases.

The effects from diabetes on the health care system are startling. In the U.S., the cost of hospitalizations, supplies, lost work, disability payments and premature deaths from diabetes reached more than $245 billion in 2012 alone. In addition, the long-term complications associated with diabetes, particularly when poorly managed, can lead to serious financial and human consequences. Serious diabetes-related complications, including cardiovascular disease, kidney disease, nerve damage, blindness, circulatory problems (which can lead to amputations), stroke, heart disease and pregnancy complications, are estimated to cost more than $176 billion annually. Some health maintenance organizations estimate that while only 3.1% of their covered patients have diabetes, diabetic patients account for over 15% of their total healthcare costs.

Research conducted by the National Institute of Health has shown that if people with diabetes closely monitor and control their blood glucose (Bu levels, they will enjoy significant health benefits. Consistent management of diabetes, which includes diet, exercise and aggressive monitoring and control of blood glucose levels, can lessen the risk of serious complications and potentially reduce some diabetes-related conditions by more than half.

The study further revealed that active management of diabetes could, among other benefits, reduce eye disease by up to 76%, reduce kidney disease by up to 50% and reduce nerve disease by up to 60%. Furthermore, treatment regimens necessitate tightly controlled glucose levels, which inherently cause an increased risk of more frequent hypoglycemic episodes. A very real issue facing many diabetics is the fear and possibility of falling into a hypoglycemic coma, or experiencing other diabetic emergencies, without external assistance. Likewise, the fear of a diabetic emergency in a child or other dependent confronts many parents and guardians of diabetic individuals. The possibility of a diabetic emergency hinders both the diabetic individual and the guardian from leading active, independent lifestyles. Thus, what are needed are improved diabetes management systems and methods.

### SUMMARY

In a first aspect, apparatus for diabetes management is provided. The apparatus includes a portable diabetes management system (DMS) device. The DMS device includes a processor, a data storage device, a touchscreen display, wireless communications facilities, a pattern recognition engine stored in the data storage device and executable in the processor, and a user interface structure stored in the data storage device and executable in the processor. The user interface structure includes a plurality of user interface displays configured to be displayed on the touchscreen display, wherein one of the plurality of user interface displays includes a listing of a selectable subset of a plurality of different patterns based on blood glucose measurement data received by the DMS device, the selectable subset of patterns based upon a frequency with which the different patterns are detected by the pattern recognition engine.

In a second aspect, a method for diabetes management is provided. The method includes receiving blood glucose measurements at a portable wireless device from a blood glucose meter; storing the blood glucose measurements in a data storage device of the portable wireless device; recognizing one or more patterns with a processor of the portable wireless device based on the blood glucose measurements, wherein the processor executes a pattern recognition engine stored in the data storage device; and prompting a user via a user interface of the portable wireless device to take an action in response to the recognizing of the one or more of the patterns.

Numerous other aspects are provided in accordance with these and other aspects of the invention. Other features and aspects of the present invention will become more fully apparent from the following detailed description, the appended claims and the accompanying drawings.

### BRIEF DESCRIPTION OF THE DRAWINGS

FIG. 1 is a schematic block diagram depicting an example system according to embodiments of the present invention.

FIG. 2 is a schematic block diagram depicting an example apparatus according to embodiments of the present invention.

FIG. 3 is a tabular representation of an example diabetes management system (DMS) database according to embodiments of the present invention.

FIG. 4 is a screenshot of an example interface for selecting pattern types according to embodiments of the present invention.

FIG. 5A is a screenshot of an example interface for selecting a test frequency goal according to embodiments of the present invention.

FIG. 5B is a screenshot of an example interface for presenting and managing detected patterns according to embodiments of the present invention.

FIG. 6A is a screenshot of an example interface for presenting details of a detected “improved” pattern according to embodiments of the present invention.

FIG. 6B is a screenshot of an example interface for presenting details of a detected “worked on” pattern according to embodiments of the present invention.

FIG. 7 is a block diagram illustrating an example structure of a system software architecture according to embodiments of the present invention.

FIG. 8 is a flowchart depicting an example method for an Information and Motivational Behavior (IMB) module according to embodiments of the present invention.

FIG. 9 is a block diagram depicting an example workflow according to embodiments of the present invention.

FIG. 10 is block diagram illustrating details of an example Information and Motivational Behavior (IMB) module portion of a system software architecture according to embodiments of the present invention.

FIGS. 11 through 31 are flowcharts depicting various example methods of detecting patterns according to embodiments of the present invention.

FIGS. 32 through 35 are flowcharts depicting various example methods of transitions within Pattern Map Flows according to embodiments of the present invention.

### DESCRIPTION

For the purposes of promoting an understanding of the principles of embodiments of the invention, reference will now be made to the examples illustrated in the drawings and specific language will be used to describe the same. It will nevertheless be understood that no limitation of the scope of the invention is thereby intended, and any alterations and further modifications in the illustrated embodiments, and any further applications of the principles of the invention as illustrated therein as would normally occur to one skilled in the art to which the invention relates are contemplated herein.

Embodiments of the present invention provide systems, apparatus and methods for an improved diabetes management system (DMS). In order to manage their disease, people with diabetes (each a “PWD”) often test their blood glucose level multiple times per day and keep track of their carbohydrate intake, exercise, and insulin doses. To record these metrics and ensure they keep to their testing regimen, PWDs can manually track information on paper, on a computer, or on a smart device. However, it is useful to identify numerous patterns related to blood glucose readings over time to help PWDs better manage their health. Examples of such patterns include critical low meter reading, critical high meter reading, test frequency low, test frequency fair, test frequency good, testing mostly same time, high for the time of day, low for the time of day, best time of day, fasting high, fasting low, pre-lunch high, pre-lunch low, pre-throughout high, pre-dinner low, post dinner high, post dinner low, running high, running low, day of week low, day of week high, etc. The sheer number of useful patterns and the data involved with recognizing these patterns is overwhelming and it is not practical for a user to manually track all the information needed to identify occurrences of the patterns, much less actually detect occurrences in real time. Thus, embodiments of the present invention automate data capture and storage as well as pattern recognition. Further, many patterns can occur and be detected within a relatively short time span which can result in an overwhelming number of notices and reminders being available for presentation to the user. Embodiments of the present invention provide interface facilities and features to help the user manage, filter, and prioritize notices and reminders.

Embodiments of the present invention include software applications and systems adapted to provide an enhanced system for managing diabetes. Using a portable, wireless device such as, for example, a smartphone, in communication with a blood glucose meter (BG meter or BGM), embodiments of the present invention include a software application (e.g., a DMS app) operable to receive blood glucose measurements and store the measurements in a DMS database for correlating the measurements with user activities and patterns. Some embodiments of the present invention enable PWDs to become more active in their diabetes management by enabling receipt of reminders from the DMS App. Some PWDs may be less active in their management because they are forgetful or unmotivated. Providing a facility to receive reminders to test their blood glucose, take their medication, or perform other diabetes management-related tasks, can help a PWD become more active and involved in their health management.

According to embodiments of the present invention, a user can set reminders within a DMS App that, when triggered, will prompt a user to test their blood glucose level, take their medication, log their activity, log their carbohydrate intake and/or any other diabetes-related task. The reminders can be automatically triggered based upon patterns being recognized by the DMS application in response to BGM data being received in the user's DMS device. In other words, in response to the DMS application recognizing one or more patterns in the BGM data (e.g., a set of patterns that collectively indicate a particular status of condition), the DMS application can generate and present recommendations, reminders, and/or warnings to the user. In some embodiments, the reminders presented can be prioritized based upon user defined and/or medical priorities. Higher priority reminders can be presented ahead of, with more intensity than (e.g., with larger text, with brighter highlighting, with different colors, with sound, etc.), and/or more frequently than lower priority reminders. In some embodiments, the frequency with which a particular reminder is presented to the user can be restricted or limited. For example, if a reminder to record exercise is presented to the user in response to the user not recording any exercise for three days, a subsequent reminder that is triggered three days later for the same reason may be suppressed. In this manner, overloading the user with redundant reminders is avoided.

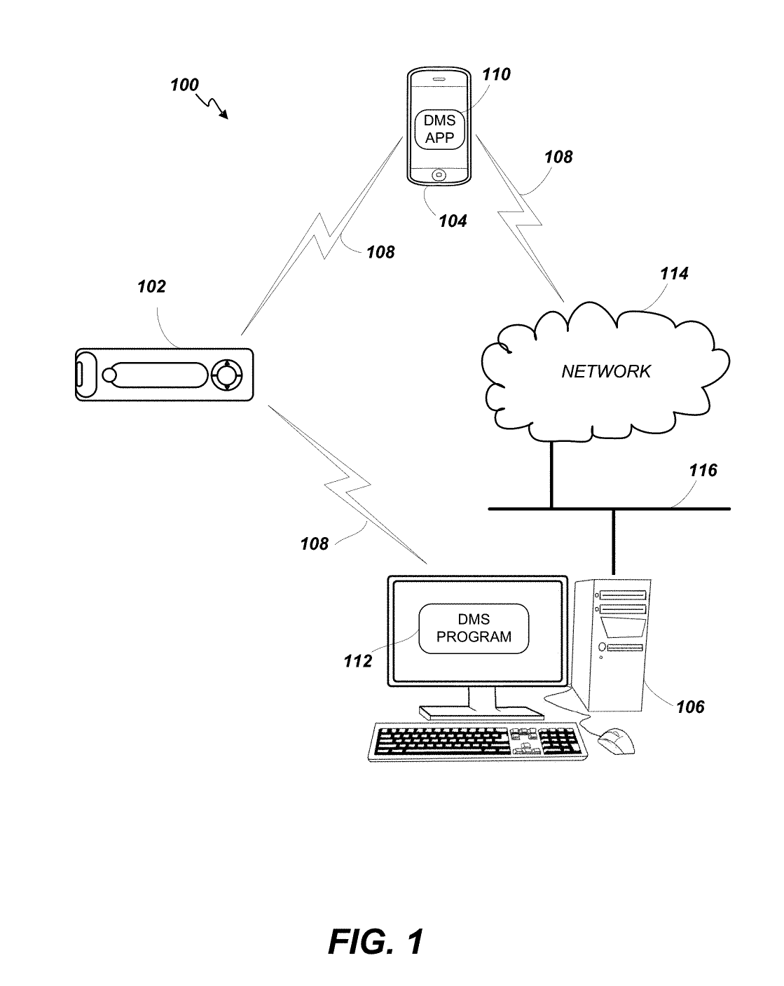

Turning now to FIG. 1, an example of a DMS 100 is depicted. The DMS 100 includes a BGM 102 that is adapted to couple to a DMS device 104 (e.g., a smart phone, tablet, smart watch, etc. operative to execute a DMS App 110) and/or a computer 106 operative to run a DMS program 112. The BGM 102 and the DMS device 104 are operated by a user (e.g., a PWD) using the DMS 100 to help improve their management of their diabetes. The DMS device 104 and the computer 106 can be coupled to the BGM 102 wirelessly (e.g., via a wireless signal protocol 108 such as Bluetooth) or via a wired connection (e.g., via a universal serial bus (USB) connection).

In some embodiments, a health care provider (HCP) or user can operate the computer 106 to receive glucose readings data from the BGM 102 and other data from the DMS device 104 via a network 114 (e.g., the Internet). In some embodiments, the computer 106 can receive glucose readings data directly from the BGM 102 via a wired, wireless, or and any other practicable means (e.g., a memory card exchange). The computer 106 can couple to the network 114 via a wired connection (e.g., via Ethernet 116) or via any other practicable means. Likewise, the DMS device 104 can couple to the network 114 via a wireless signal protocol 108 (e.g., Wi-Fi) or via any other practicable means.

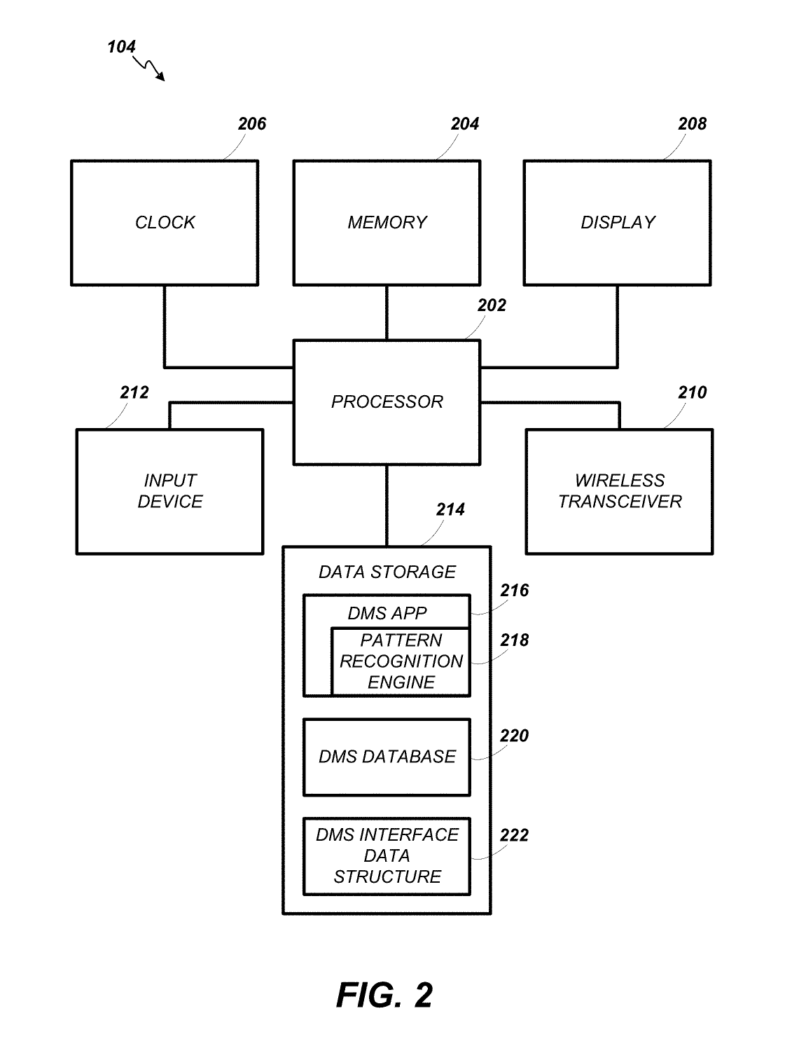

Turning now to FIG. 2, details of an example DMS device 104 are depicted. Note that in some embodiments, the DMS device 104 can be implemented on a computer 106 and the computer 106 can be a portable, wireless device (e.g., a laptop, tablet PC, etc.). A DMS device 104 can include a processor 202 coupled to a memory 204 for storing instructions executable on the processor 202. The memory 204 can also be used to cache data retrieved from or to be stored in a data storage device 214. The processor 202 can be coupled to a clock 206 (e.g., a clock generator module, an oscillator, etc.) for generating date and timestamp data to associate with BGM and/or other data.

The processor 202 can be coupled to a display 208 that can include any number of output devices (e.g., such a display, an audio speaker, haptic devices, a vibrator, light emitting diodes (LEDs), a printer, audio output, USB and LAN ports, and the like). The display 208 can be used to communicate with the user to present reminders as well as for conventional output functions.

The processor 202 can be coupled to a wireless transceiver 210 that can include cellular communications facilities and two-way radio signal communication facilities such as Wi-Fi, Bluetooth, and other communications modules. In other words, the wireless transceiver 210 can include any type of device and/or software that is able to communicate across the network 114. For instance, the wireless transceiver 210 can include a cellular communication type device, a Wi-Fi type device, and/or an infrared port, to name just a few.

The processor 202 can be coupled to input device 212 which can, for example, include any number of input devices (e.g., such as a touch screen, “soft” programmable buttons/keys, hardware buttons and switches, keyboard, optical and magnetic readers/scanners, cameras, sensors, transducers, accelerometer, microphones, audio input, USB and LAN ports, and the like). The input device 212 can be used to communicate with the user to set reminders or other parameters as well as for conventional input functions.

The processor 202 can be coupled to a data storage device 214 such as non-volatile memory that allows persistent storage of data structures, data, and instructions that can be loaded into the memory 204 for use/execution by the processor 202. The data storage device 214 can be implemented using one or more solid state drives, hard drives, memory cards, or the like. The data storage device 214 includes a data structure that can include a DMS App 216 (that in some embodiments includes an integrated pattern recognition engine 218), a DMS database 220, and a DMS interface data structure 222.

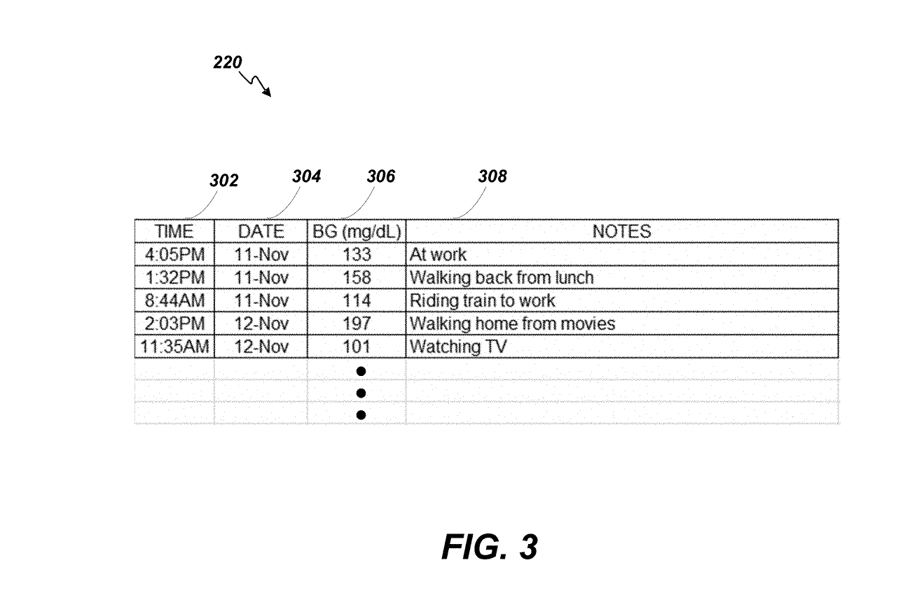

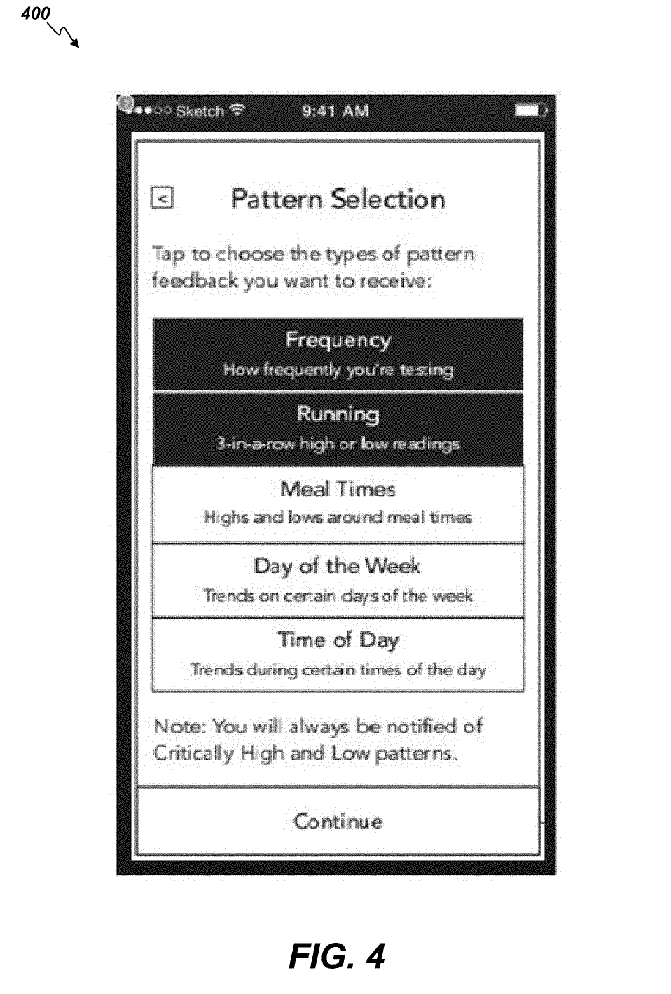

The DMS App 216 implements the methods and processes described herein. The pattern recognition engine 218 is used by the DMS App 216 to implement detection of behaviors (e.g., via recognizing patterns in the captured BGM, user entered, and other data) that result in helpful or hurtful events (e.g., good or poor blood glucose control). An example of a pattern recognition system is disclosed in U.S. Pat. No. 8,758,245 to Ray et al. which is incorporated herein for all purposes. An example of a DMS database 220 is described below with respect to FIG. 3. A DMS interface data structure 222 can include a plurality of user interface displays that are related by use flows between the displays. In other words, each user interface display is linked to and/or reachable via at least one other user interface display or is presented as the result of a pattern being detected or some other related triggering event. Examples of user interface displays are depicted in FIGS. 4 through 6B and described below.

Turning now to FIG. 3, an example of a DMS database 220 is depicted in tabular form. Note that the format of the particular example depicted is merely illustrative of one possibility. Many alternative data arrangements and database types could be used. Any format or database type practicable to implement the data structure and relationships depicted could be used. Note also that only a limited number of entries are shown in the example and that in an actual implementation, there may be many more entries (e.g., thousands of rows).

Each entry in the DMS database 220 shown can include a time field 302, a date field 304, a blood glucose level field 306, and a notes field 308. The time field 302 is adapted to store data representative of a timestamp that indicates the time a blood glucose reading associated with the entry occurred. The date field 304 is adapted to store data representative of a date-stamp that indicates the date the blood glucose reading associated with the entry occurred.

The blood glucose level field 306 is adapted to store data representative of a blood glucose level of the blood glucose reading associated with the entry. The notes field 308 is adapted to store data representative of information provided by the user and associated with the entry.

In some embodiments, many additional fields can be included in the DMS database 220. For example, a medication dosage field, a food intake field, a carbohydrates consumed field, an exercise performed field, and the like can be included.

FIG. 4 is a screenshot of an example interface display 400 for selecting pattern types. The user is presented with a list of pattern types that can be selected via depressing the indicated areas on the interface display 400. The information is stored and the selected types of patterns are used to determine which patterns are presented to the user when later detected by the DMS App.

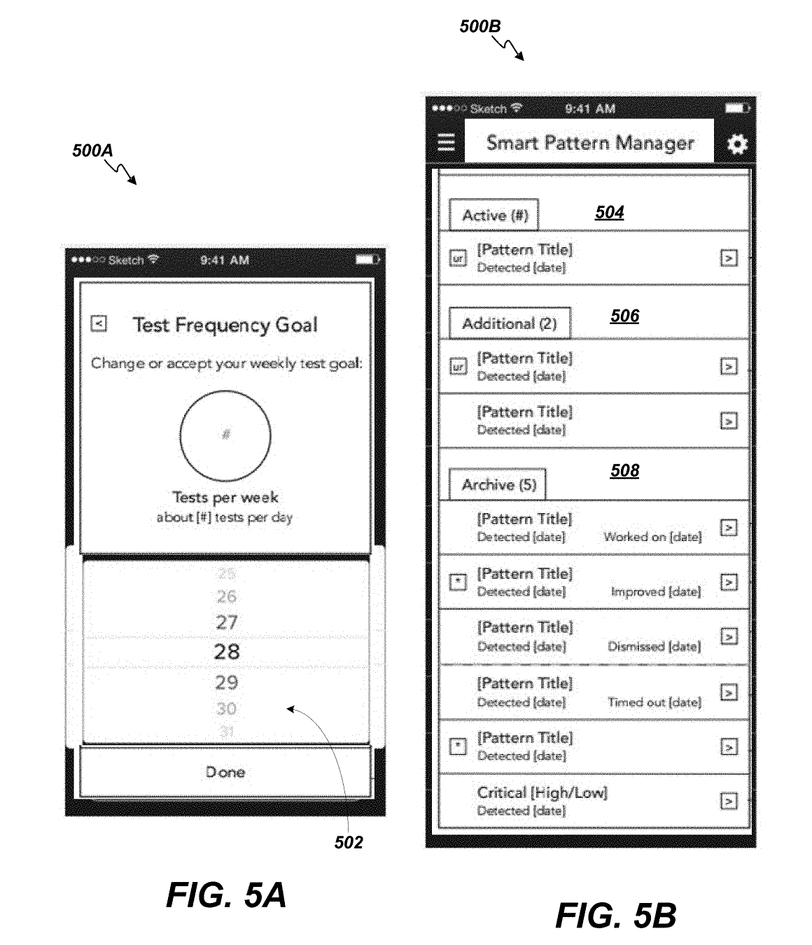

FIG. 5A is a screenshot of an example display interface 500A for selecting a test frequency goal. A scrollable window 502 allows the user to pick the number of tests per week that the DMS App will encourage the user to perform. For example, the user will be reminded to test more frequently if patterns are detected that indicate the user is falling short of the selected test frequency.

FIG. 5B is a screenshot of an example Pattern Manager display interface 500B for presenting and managing detected patterns according to embodiments of the present invention. The Pattern Manager display interface 500B includes areas for interactive lists of Active 504, Additional 506, and Archived 508 detected patterns. These categories of detected patterns are discussed in more detail below.

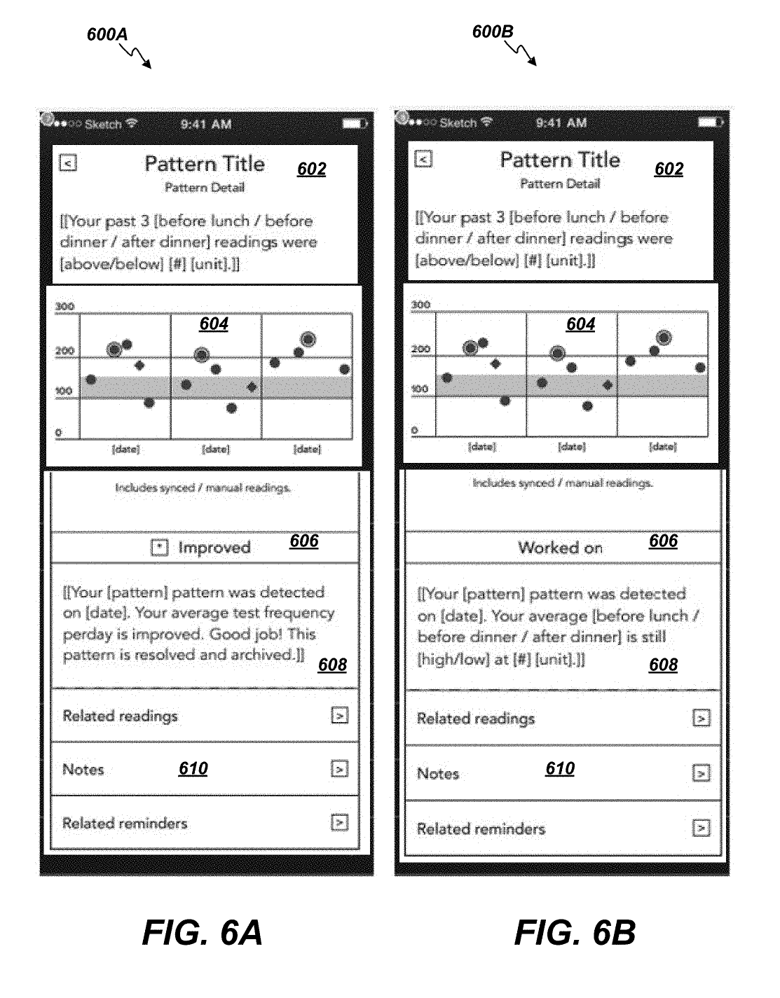

FIG. 6A is a screenshot of an example display interface 600A for presenting details of a detected “improved” pattern and FIG. 6B is a screenshot of an example display interface 600B for presenting details of a detected “worked on” pattern. These display interfaces 600A, 600B are examples of the details presented when the user selects a detected pattern from the Pattern Manager display interface 500B of FIG. 5B. The display interfaces 600A, 600B include a summary area 602, a graph area 604, a status area 606, an explanation area 608, and a “further links” area 610.

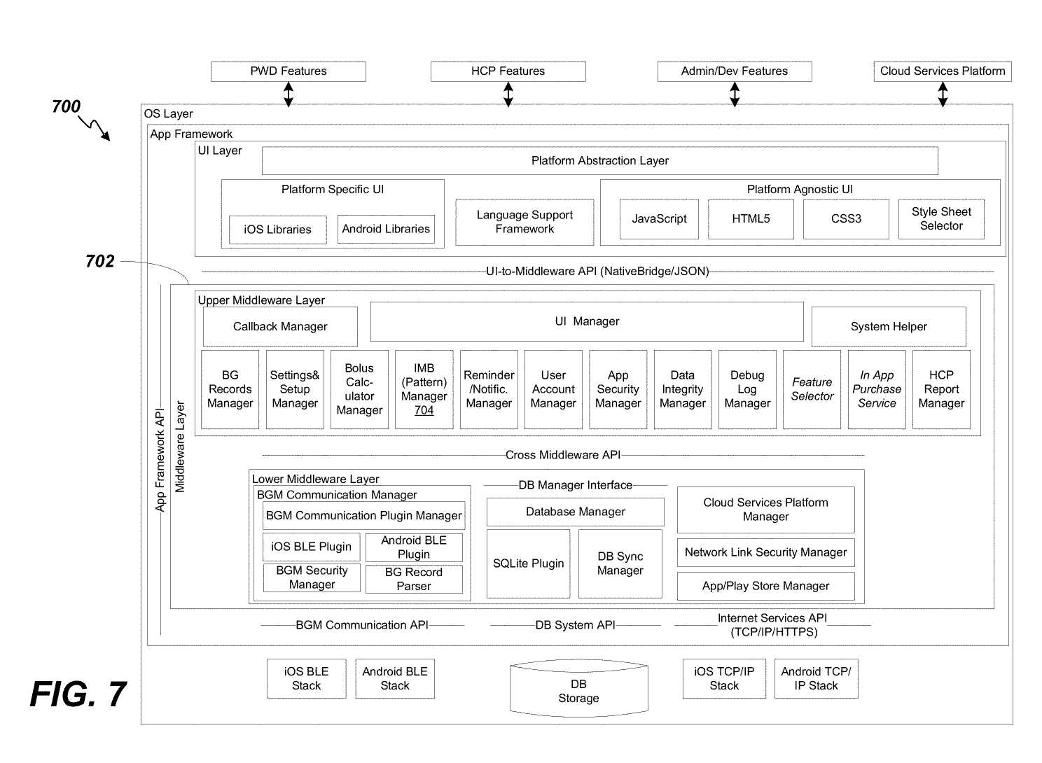

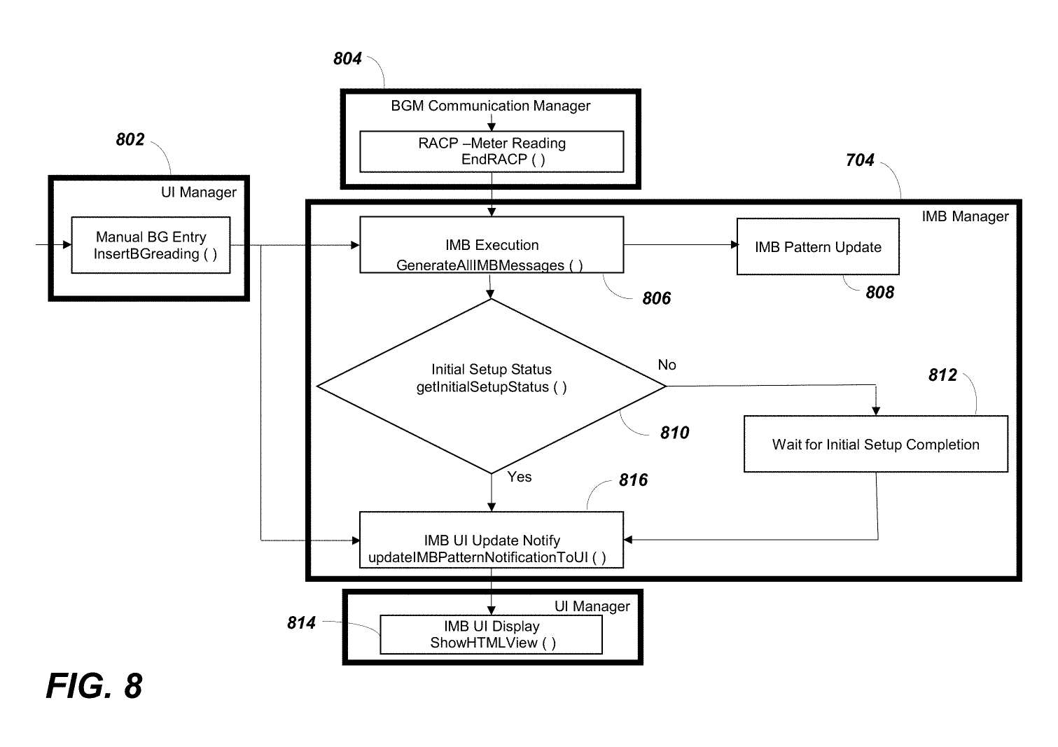

In alternative embodiments, a DMS application can be implemented as part of an integrated system architecture 700 as illustrated in FIG. 7. Residing within a middleware application program interface 702, an Information and Motivational Behavior (IMB) manager 704 can implement the functionality described above. As shown in the flowchart 800 of FIG. 8, the IMB manager 704 can receive BG information either manually through the user interface manager 802 or via the BGM communication manager 804 (e.g., wirelessly). IMB Execution commences with the generation of IMB (e.g., reminder) messages (806) and updates to the stored IMB patterns (808). Based on the initial setup status (810) the IMB manager either waits for setup completion (812) or sends an update notification to the IMB user interface display 814 of the user interface manager 802 (816).

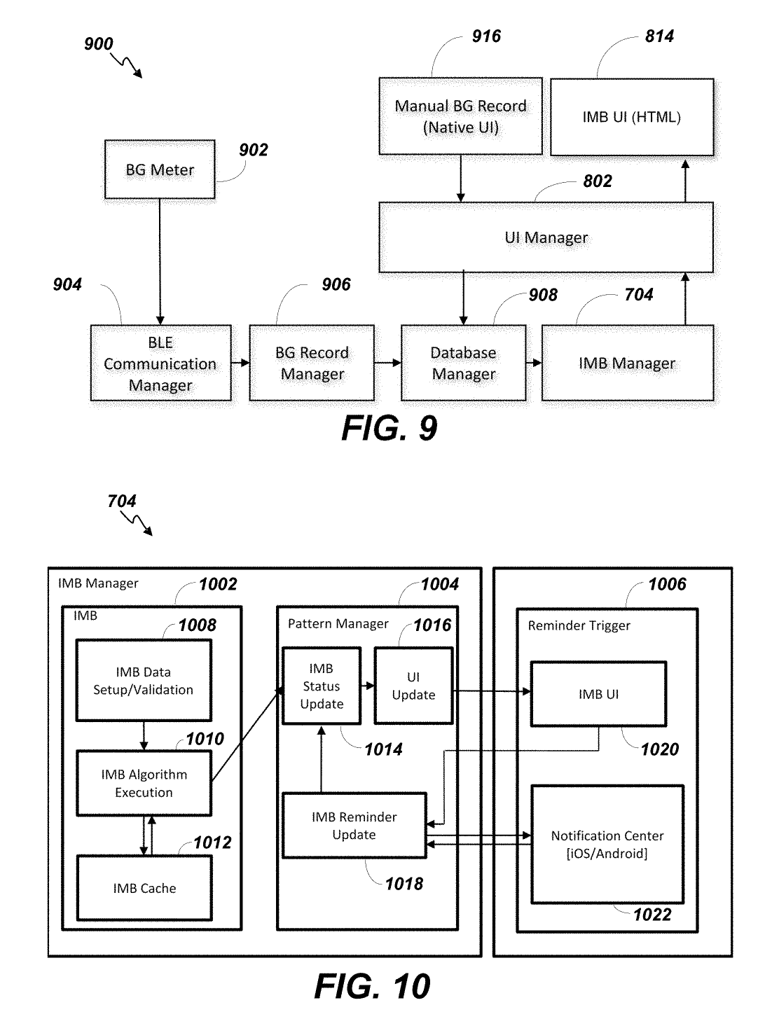

FIG. 9 is a block diagram that depicts the IMB workflow 900. The BG meter 902 can provide a BG reading to the communication manager 904 (e.g., via Bluetooth Low Energy (BLE) protocol) within the application when the BG meter 902 is connected. The BG Record Manager 906 module will identify the BLE data (e.g., whether incoming data identifies BG reading, a meal marker, or setting data), parse and reform the data as a corresponding record (e.g., a BG/Meal Marker record, etc.) and send it to database manager 908 to be stored in the database. The database manager 908 will store the BG/Meal Marker/Device Setting data into the database (e.g., an SQLite database) and will perform data read operations from the database. IMB manager 704 executes the IMB module whenever a new BG reading arrives and IMB data will be stored in the database through the IMB pattern Manager and an IMB notification will be sent to IMB user interface 802 for display. The user interface Manager 802 is a gateway for the middleware 702 since all user interface operations (e.g., data reads/writes) to the middleware 702 happen through this module. In some embodiments, IMB notifications can be sent to the HTML level through this module in JSON format. This module gets data from the database, formats the data (e.g., in JSON), and sends the formatted data to the user interface. The Manual BG Records module 916 can also generate BG data records (e.g., from a BG data storage application) that are similar to BG meter records but instead of a BG meter determining BG readings from strip measurements, the data records are “generated” from the application. In the case of manual entry, the Manual BG Records module 916 directly interacts with the database Manager 908 (e.g., to store the manual entry in the database) through user interface Manager 802.

FIG. 10 depicts in more detail the structure and components of the IMB manager 704. In some embodiments, the IMB manager 704 includes an IMB module 1002 and a Pattern Manager module 1004. The IMB manager 704 also interacts with the Reminder Trigger module 1006.

The IMB module 1002 includes three sub-modules: the IMB Data Setup/Validation sub-module 1008, the IMB Algorithm Execution sub-module 1010, and the IMB Cache sub-module 1012. The IMB Data Setup/Validation sub-module 1008 is employed whenever a new BG reading is received, regardless of originating from the BG meter or from manual entry. The IMB module 1002 will enter setup mode, validate the data, and make the decision whether the IMB algorithm should be executed or not. Setup or validation is done via first getting target range values, next resetting the IMB cache 1012, checking the IMB Execution eligibility status based on the current/last executed BG timestamp, and then checking and updating the pattern “timed out” status for already detected IMB pattern in Pattern Manager module 1004. The IMB Algorithm Execution sub-module 1010 is responsible for execution of the IMB algorithm; updating of the IMB cache 1012 for UI notification; and updating/inserting new detected patterns into the Pattern Manager module 1004. The IMB Cache sub-module 1012 functions as a local buffer and holds information about the IMB pattern currently being detected. The information can include the IMB ID and whether the pattern is a delayed pattern or not.

The Pattern Manager module 1004 includes three sub-modules: the IMB Status Update sub-module 1014, UI Update sub-module 1016, and the IMB Reminder Update sub-module 1018. IMB status is an important property of an IMB pattern. The Pattern Manager module 1004 updates the IMB pattern status. The IMB Status Update sub-module 1014 can include several pieces of status information. For example, the information can include New Pattern detected information, a Pattern category update (e.g., Active/Archive), a Pattern state update (e.g., Read/Unread), and a Pattern status update (e.g., New/Started/On-Hold Int/Working/On-Hold Cau/Rem-Setup/Dismissed_Rem/Finished/Dismissed_Setup/Improved/Invalid/Followed/Needs Improvement/Overcorrected/Timed-Out). In some embodiments, the New status may be assigned to the newly detected pattern before a user is presented with a Pattern Interface screen; the Started status may be assigned to a pattern if a user chooses to move forward with an IMB Flow on Pattern Detection screen; the On-Hold Int status may be assigned to a pattern if a user closes a Pattern Interface screen; the Working status may be assigned to a pattern if a user chooses to move forward with a IMB Flow on Possible Causes screen; the On-Hold Cau status may be assigned to a pattern if a user closes a Possible Causes screen; the Rem-Setup status may be assigned to a pattern if a user chooses to move forward with an IMB Flow on Need Reminder screen; the Dismissed_Rem status may be assigned to a pattern if a user chooses not to move forward with the IMB flow in “Need Reminders” screen; the Finished status may be assigned to a pattern if a user finishes and confirms setting up a reminder during IMB flow for all the other patterns; the Dismissed_Setup status may be assigned to a pattern if a user doesn't confirm a reminder setup (i.e., closes a “Reminder Setup” screen); the Improved status may be assigned to a pattern in the following 2 cases: (1) after getting positive feedback after a follow-up and (2) if new or changed records contribute to resolving the pattern; the Followed status may be assigned to a pattern after getting negative feedback after the follow-up; the Needs Improvement status may be assigned to Critical patterns if before a pattern improvement or before a pattern timing out; the Overcorrected status may be assigned to a Critical High or Critical Low pattern if after retesting a value of the BG record turns out to be Critically Low or Critically High respectively; and the Timed-Out status may refer to each pattern having a pre-determined time period in which a time out status will be assigned. Once a pattern has timed out, it moves to the Archive section (described below). An Active pattern may time out if not improved in a pattern specific period.

The UI Update sub-module 1016 is responsible for presenting detected IMB patterns in the UI. If a reminder has been created during IMB pattern flow, then the IMB Reminder Update sub-module 1018 performs an update of the Reminder ID for the corresponding IMB pattern and an update of the IMB reminder triggered status. The Reminder Trigger module 1006 represents a UI 1020 or native 1022 (e.g., Android or IOS) notification center which initiates the creation of a reminder, triggers a reminder, and updates a reminder's status.

The IMB module 1002 can be configured to recognize and manage any number of patterns. The following twenty-one patterns are described in detail below: critical high meter reading, critical low meter reading, test frequency low, test frequency fair, test frequency good, testing mostly same time, high for the time of day, low for the time of day, best time of day, fasting high, fasting low, pre-lunch high, pre-lunch low, pre-throughout high, pre-dinner low, post dinner high, post dinner low, running high, running low, day of week low, and day of week high.

The IMB module 1002 informs the user of detected patterns from the history of the user's BGM data (e.g., records from the DMS database 220) and provides mechanisms to be used for better diabetes management. In some embodiments, IMB pattern detection will generally look at 14 days of BGM data history. Note however that some of the patterns consider up to 21 days of history and some only use a single BG Reading. If a note is entered by the user on any of the IMB interface screens where applicable, that note is saved in the DMS database and is available in the Edit View/Notes tab for the associated BG reading.

The DMS App 216 initiates running IMB algorithms by the IMB Algorithm Execution sub-module 1010 (e.g., triggering IMB Patterns) when one or more new BG records are acquired by the DMS App 216 or when an existing (e.g., previously acquired) BG record is modified. Each of the IMB algorithms accepts similar input which includes a set of BG records, each with a Blood Glucose Reading Value (referred to herein as BGRecordValue in algorithm description) and a Blood Glucose Reading Timestamp (referred to herein as BGRecordTimeStamp). In addition, each of the IMB algorithms accepts additional inputs such as, for example, threshold and/or target values. Each of the example IMB algorithms described herein are used to trigger a corresponding pattern based on the BG readings and time.

Each of the algorithms has the same output type: a BOOLEAN value of “0” if the associated pattern was not detected and “1” if the associated pattern was detected. As discussed above, the output of the IMB algorithms is one of the inputs for the Pattern Manager module 1004. If a certain pattern is detected, then the Pattern Manager module 1004 triggers notification of a new pattern. After acknowledging this notification, a series of UI messages (e.g., screen displays) referred to as IMB Pattern Maps, will be presented to the user one after another depending on the level of choices the user makes on each screen. IMB patterns can be divided into critical IMB patterns and non-critical IMB patterns.

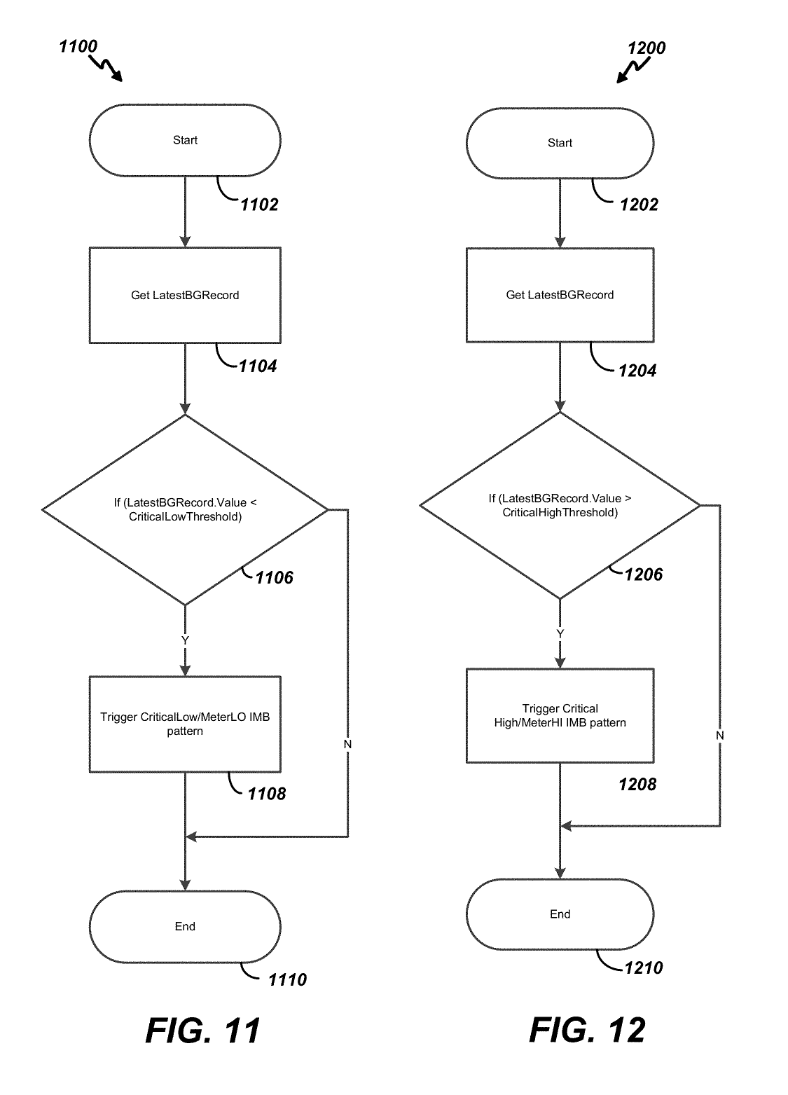

An example of an algorithm or method 1100 for recognizing a critical low pattern is illustrated as a flowchart in FIG. 11. When a new BG record is acquired or a previously acquired record is modified, the DMS App will trigger this pattern if the BG record value is below the value specified as CriticalLowThreshold. The method 1100 starts when a new BG record is acquired or a previously acquired record is modified (1102). The latest BG record is retrieved (1104) and it is determined if the BG value is less than the stored parameter CriticalLowThreshold (1106). If so, the critical low pattern is triggered (i.e., detected), the Pattern Manager module 1004 is so notified (1108) by the IMB Algorithm Execution sub-module 1010 and the method 1100 completes (1110). Otherwise, the method 1100 simply completes (1110).

An example of an algorithm or method 1200 for recognizing a critical high pattern is illustrated as a flowchart in FIG. 12. When a new BG record is acquired or a previously acquired record is modified, the DMS App will trigger this pattern if the BG record value is above the value specified as CriticalHighThreshold. The method 1200 starts when a new BG record is acquired or a previously acquired record is modified (1202). The latest BG record is retrieved (1204) and it is determined if the BG value is greater than the stored parameter CriticalHighThreshold (1206). If so, the critical high pattern is triggered (i.e., detected), the Pattern Manager module 1004 is so notified (1208) and the method 1200 completes (1210). Otherwise, the method 1200 simply completes (1210).

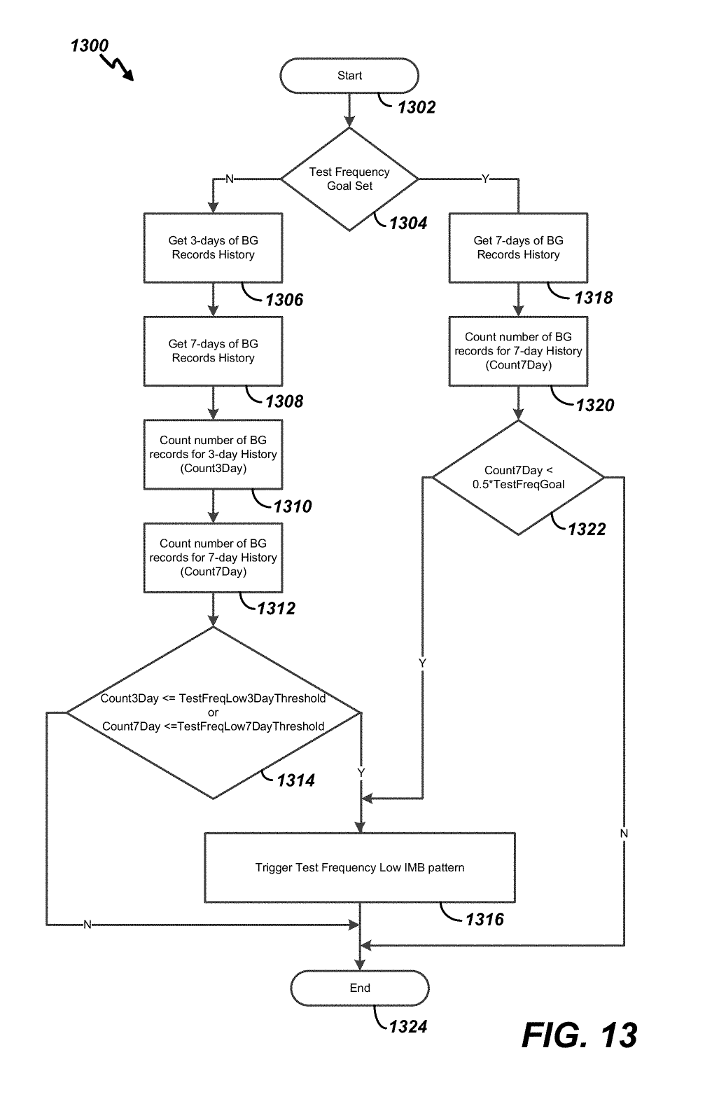

An example of an algorithm or method 1300 for recognizing a test frequency low pattern is illustrated as a flowchart in FIG. 13. The method 1300 starts when a new BG record is acquired or a previously acquired record is modified (1302). When a new BG record is acquired or a previously acquired record is modified, the DMS App will trigger this pattern if it detects that User's testing frequency is below the threshold value specified as TestFreqLow3DayThreshold (or TestFreqLow7DayThreshold if the user has set a test frequency goal) based on the following algorithm. It is first determined if the user has set a test frequency goal (1304). If TestFreqGoalSet=0, then 3-days of BG records history (e.g., from the current time back 72 hours) are retrieved (1306) and 7-days of BG records history (e.g., from the current time back 168 hours) are retrieved (1308). Then the number of BG Readings per day in the 3-day history (Count3Day) is counted (1310) and the number of BG Readings per day in the 7-days history (Count7Day) is counted (1312). It is then determined if either Count3Day<=TestFreq3DayLowThreshold or Count7Day<=TestFreq7DayLowThreshold (1314). If so, then the pattern is triggered (1316). Otherwise the method 1300 simply ends without triggering the pattern (1324). If TestFreqGoalSet=1, then 7-days of BG records history (e.g., from the current time back 168 hours) is retrieved (1318). The number of BG Readings per day in the 7-day history (Count7Day) is counted (1320). It is determined if Count7Day<50% of the TestFreqGoal (1322). If so, then the pattern is triggered (1316) and the method 1300 ends (1324). Otherwise the method 1300 simply ends without triggering the pattern (1324).

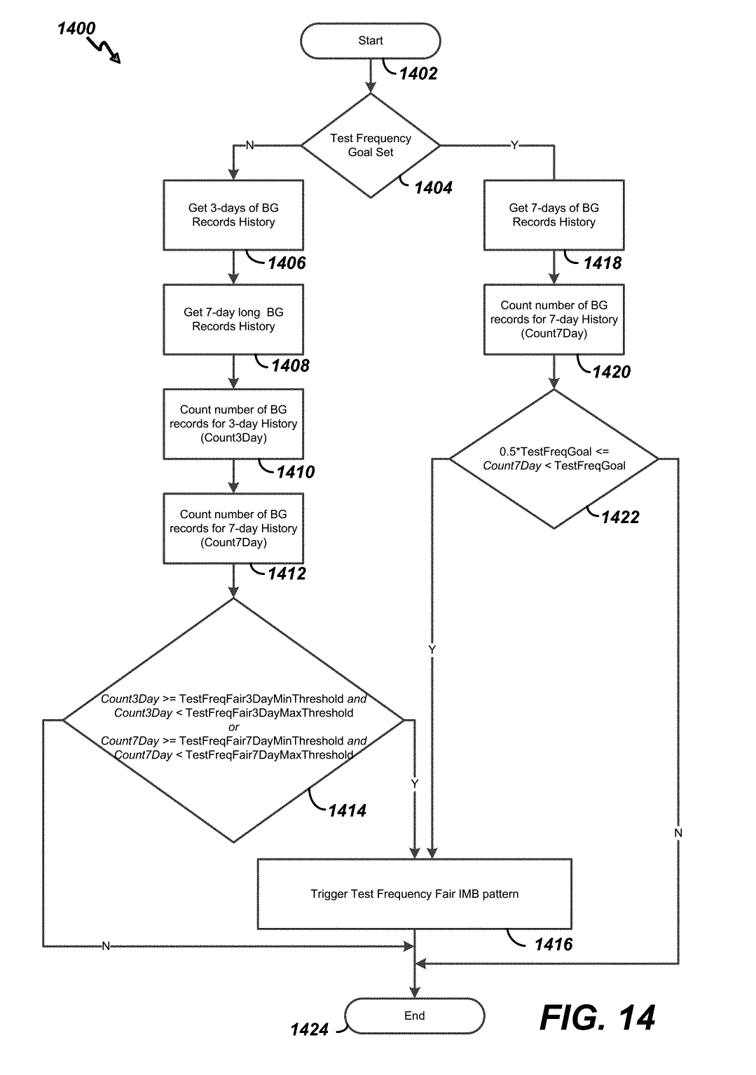

An example of an algorithm or method 1400 for recognizing a test frequency fair pattern is illustrated as a flowchart in FIG. 14. The method 1400 starts when a new BG record is acquired or a previously acquired record is modified (1402). When a new BG record is acquired or a previously acquired record is modified, the DMS App will trigger this pattern if it detects that User's testing frequency is within a target value range specified as greater than TestFreqFair3DayMinThreshold (e.g., 6) and less than TestFreqFair3DayMaxThreshold (e.g., 12) (or greater than TestFreqFair7DayMinThreshold (e.g., 14) and less than TestFreqFair7DayMaxThreshold (e.g., 28) if the user has set a test frequency goal) based on the following algorithm. It is first determined if the user has set a test frequency goal (1404). If TestFreqGoalSet=0, then 3-days of BG records history (e.g., from the current time back 72 hours) are retrieved (1406) and 7-days of BG records history (e.g., from the current time back 168 hours) are retrieved (1408). Then the number of BG Readings per day in the 3-day history (Count3Day) is counted (1410) and the number of BG Readings per day in the 7-days history (Count7Day) is counted (1412). It is then determined if either (Count3Day>=TestFreqFair3DayMinThreshold and Count3Day<TestFreqFair3DayMaxThreshold) or (Count7Day>=TestFreqFair7DayMinThreshold and Count7Day<TestFreqFair7DayMaxThreshold) (1414). If so, then the pattern is triggered (1416). Otherwise the method 1400 simply ends without triggering the pattern (1424). If TestFreqGoalSet=1, then 7-days of BG records history (e.g., from the current time back 168 hours) is retrieved (1418). The number of BG Readings per day in the 7-day history (Count7Day) is counted (1420). It is determined if Count7Day<50% of the TestFreqGoal (1422). If so, then the pattern is triggered (1416) and the method 1400 ends (1424). Otherwise the method 1400 simply ends without triggering the pattern (1424).

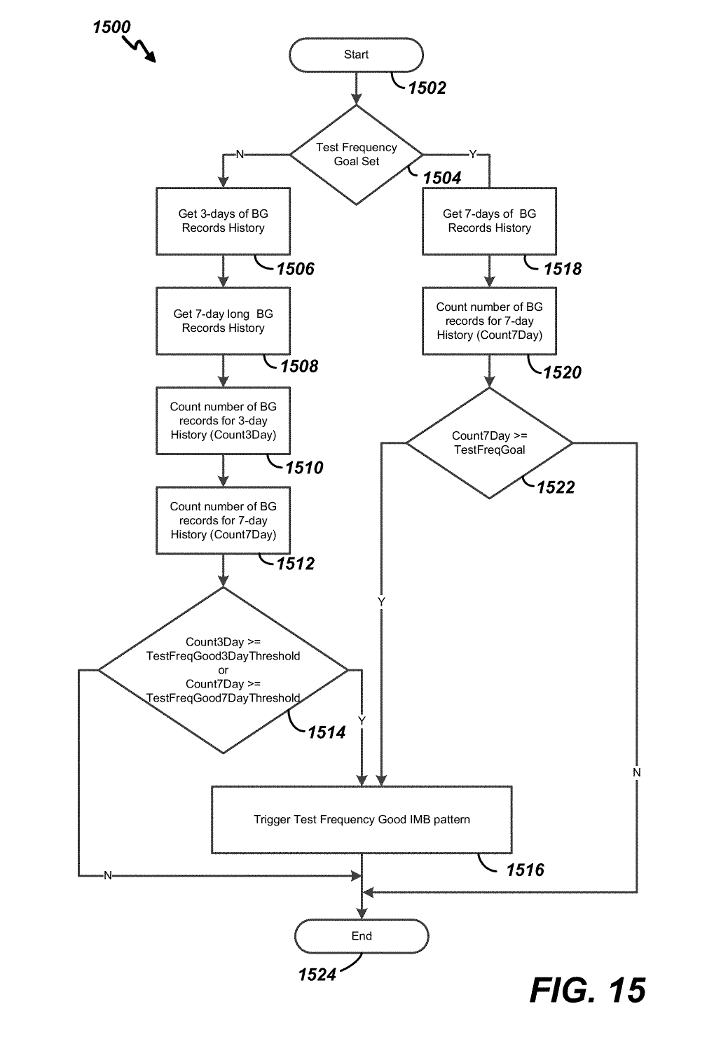

An example of an algorithm or method 1500 for recognizing a test frequency good pattern is illustrated as a flowchart in FIG. 15. The method 1500 starts when a new BG record is acquired or a previously acquired record is modified (1502). When a new BG record is acquired or a previously acquired record is modified, the DMS App will trigger this pattern if it detects that User's testing frequency is above the threshold value specified as TestFreqGood3DayThreshold (e.g., 12) (or TestFreqGood7DayThreshold (e.g., 28) if the user has set a test frequency goal) based on the following algorithm. It is first determined if the user has set a test frequency goal (1504). If TestFreqGoalSet=0, then 3-days of BG records history (e.g., from the current time back 72 hours) are retrieved (1506) and 7-days of BG records history (e.g., from the current time back 168 hours) are retrieved (1508). Then the number of BG Readings per day in the 3-day history (Count3Day) is counted (1510) and the number of BG Readings per day in the 7-days history (Count7Day) is counted (1512). It is then determined if either Count3Day>=TestFreq3DayGoodThreshold or Count7Day>=TestFreq7DayGoodThreshold (1514). If so, then the pattern is triggered (1516). Otherwise the method 1500 simply ends without triggering the pattern (1524). If TestFreqGoalSet=1, then 7-days of BG records history (e.g., from the current time back 168 hours) is retrieved (1518). The number of BG Readings per day in the 7-day history (Count7Day) is counted (1520). It is determined if Count7Day>=the TestFreqGoal (1522). If so, then the pattern is triggered (1516) and the method 1500 ends (1524). Otherwise the method 1500 simply ends without triggering the pattern (1524).

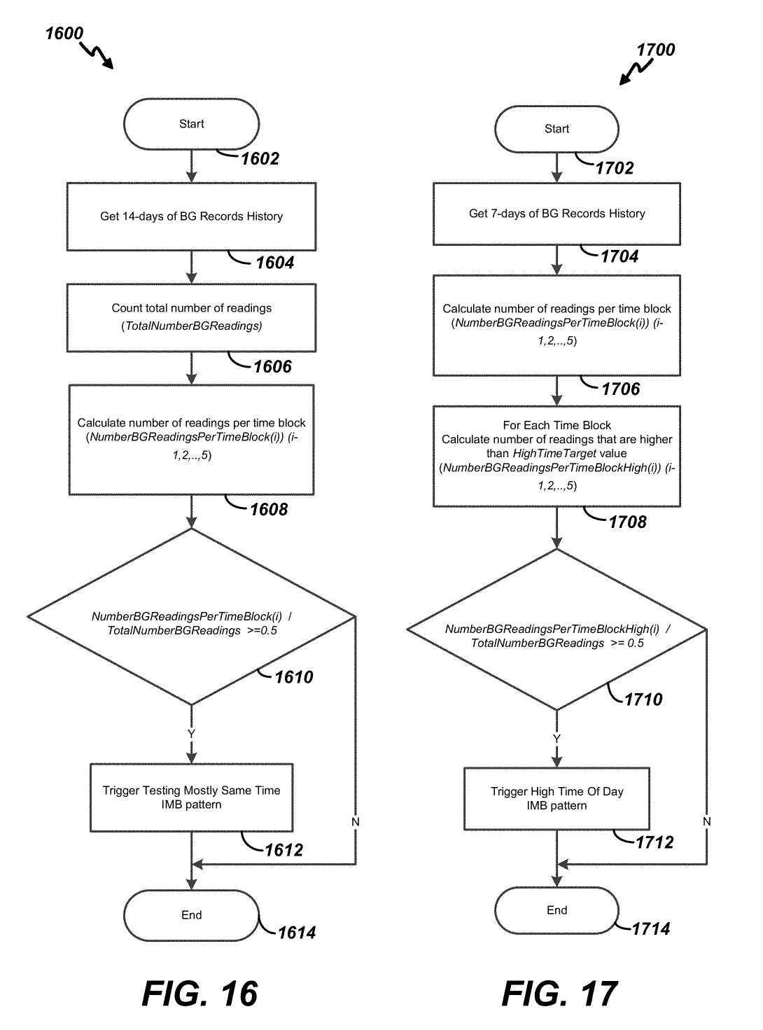

An example of an algorithm or method 1600 for recognizing a testing mostly same time pattern is illustrated as a flowchart in FIG. 16. The method 1600 starts when a new BG record is acquired or a previously acquired record is modified (1602). When a new BG record is acquired or a previously acquired record is modified, the DMS App will trigger this pattern if it detects that 50% or more of the readings (for two weeks of data going back from the time stamp of the latest BG Reading) have time stamps within a predefined “day divider” time block. The most recent two weeks of BG readings are retrieved (1604). The total number of readings (TotalNumberBGReadings) is counted for the past 14 days starting from the time stamp of the latest BG Reading (1606). Then, the number of readings per Day Divider Time Block are calculated from the entire set of readings collected within the last 14 days (NumberBGReadingsPerDayDivider(i), i=1, 2, . . . , 4) (1608). It is then determined if any of the following ratios: (NumberBGReadingsPerDayDivider(i), i=1, 2, . . . , 4)/TotalNumberBGReadings) exceeds or equals 50% (1610). If so, the pattern is triggered (1612), the day divider time blocks within which the pattern was detected are identified to the Pattern Manager module 1004, and the method 1600 completes (1614). Otherwise, the method 1600 simply ends without triggering the pattern (1614).

An example of an algorithm or method 1700 for recognizing a high time of day pattern is illustrated as a flowchart in FIG. 17. The method 1700 starts when a new BG record is acquired or a previously acquired record is modified (1702). When a new BG record is acquired or a previously acquired record is modified, the DMS App will trigger this pattern if it detects that there is a “day divider” time block in which 50% or more of the readings (for one week of data going back from the time stamp of the latest BG Reading) are higher than a predefined parameter referred to as HighTimeTarget. The most recent week of BG readings are retrieved (1704). The number of readings per day divider (NumberBGReadingsPerDayDivider(i), i=1, 2, . . . , 4) are counted for the past 7 days starting from the time stamp of the latest BG Reading (1706). Then, the number of readings per Day Divider Time Block that are higher than HighTimeTarget (NumberBGReadingsPerDayDividerHigh(i), i=1, 2, . . . , 4) (1708). It is then determined if any of the following ratios: NumberBGReadingsPerDayDividerHigh(i)/(NumberBGReadingsPerDayDivider(i), i=1, 2, . . . , 4) equals or exceeds 50% (1710). If so, the pattern is triggered (1712), the day divider time blocks within which the pattern was detected are identified to the Pattern Manager module 1004, and the method 1700 completes (1714). Otherwise, the method 1700 simply ends without triggering the pattern (1714).

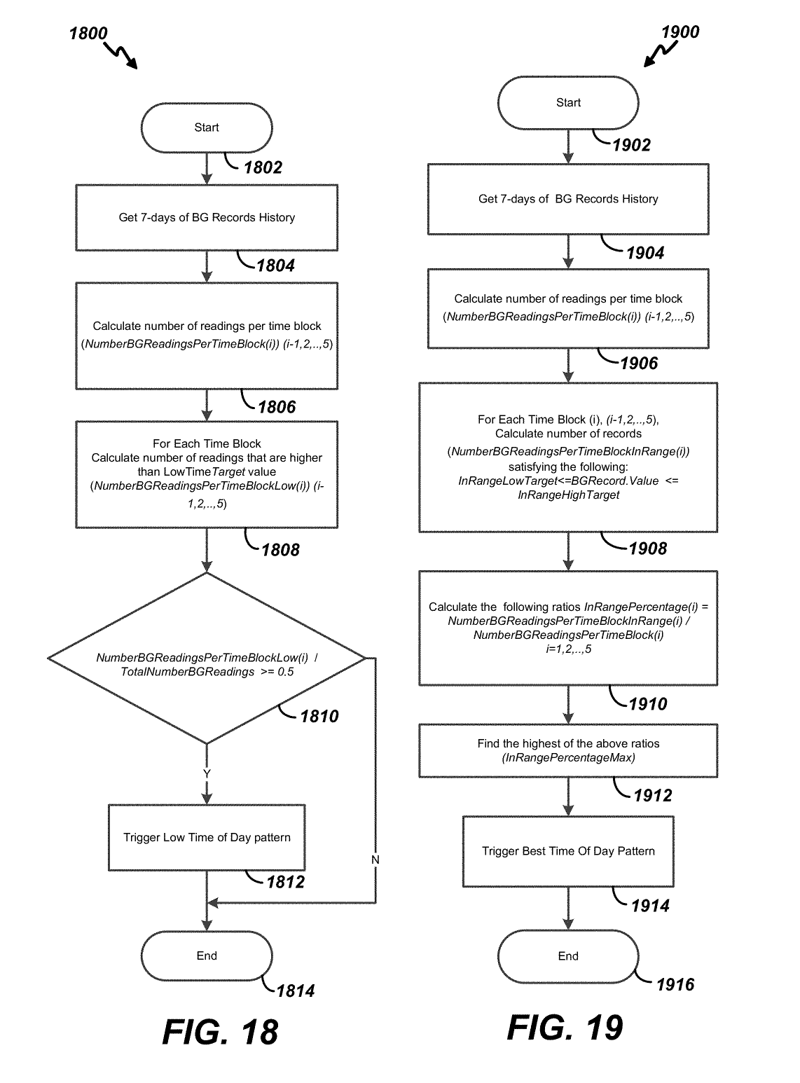

An example of an algorithm or method 1800 for recognizing a low time of day pattern is illustrated as a flowchart in FIG. 18. The method 1800 starts when a new BG record is acquired or a previously acquired record is modified (1802). When a new BG record is acquired or a previously acquired record is modified, the DMS App will trigger this pattern if it detects that there is a “day divider” time block in which 50% or more of the readings (for one week of data going back from the time stamp of the latest BG Reading) are lower than a predefined parameter referred to as LowTimeTarget. The most recent week of BG readings are retrieved (1804). The number of readings per day divider (NumberBGReadingsPerDayDivider(i), i=1, 2, . . . , 4) are counted for the past 7 days starting from the time stamp of the latest BG Reading (1806). Then, the number of readings per Day Divider Time Block that are lower than LowTimeTarget (NumberBGReadingsPerDayDividerLow(i), i=1, 2, . . . , 4) (1808). It is then determined if any of the following ratios: NumberBGReadingsPerDayDividerLow(i)/(NumberBGReadingsPerDayDivider(i), i=1, 2, . . . , 4) equals or exceeds 50% (1810). If so, the pattern is triggered (1812), the day divider time blocks within which the pattern was detected are identified to the Pattern Manager module 1004, and the method 1800 completes (1814). Otherwise, the method 1800 simply ends without triggering the pattern (1814).

An example of an algorithm or method 1900 for recognizing a best time of day pattern is illustrated as a flowchart in FIG. 19. The method 1900 starts when a new BG record is acquired or a previously acquired record is modified (1902). When a new BG record is acquired or a previously acquired record is modified, the DMS App will trigger this pattern when it finds the Day Divider time block (within one week of data starting from the time stamp of the latest BG Reading) with the highest number of in-range readings (e.g., values between predefined parameters InRangeLowTarget and InRangeHighTarget). The most recent week of BG readings are retrieved (1904). The number of readings per Day Divider from the entire set of readings collected within the last 7 days since the most recent BG Reading (NumberBGReadingsPerDayDivider(i), i=1, 2, . . . , 4) are calculated (1906). Then, for each Day Divider, the number of readings that are equal or lower than InRangeHighTarget value but equal or higher than InRangeLowTarget value (NumberBGReadingsPerDayDividerInRange(i), i=1, 2, . . . , 4) are calculated (1908). Next, the following ratios: InRangePercentage(i)=NumberBGReadingsPerDayDividerInRange(i)/NumberBGReadingsPerDayDivider(i), i=1, 2, . . . , 4 are calculated (1910). The highest of the ratios calculated above (InRangePercentageMax) is then identified for the Pattern Manager module 1004 (1912), the pattern is triggered (1914), and the method 1900 completes (1916).

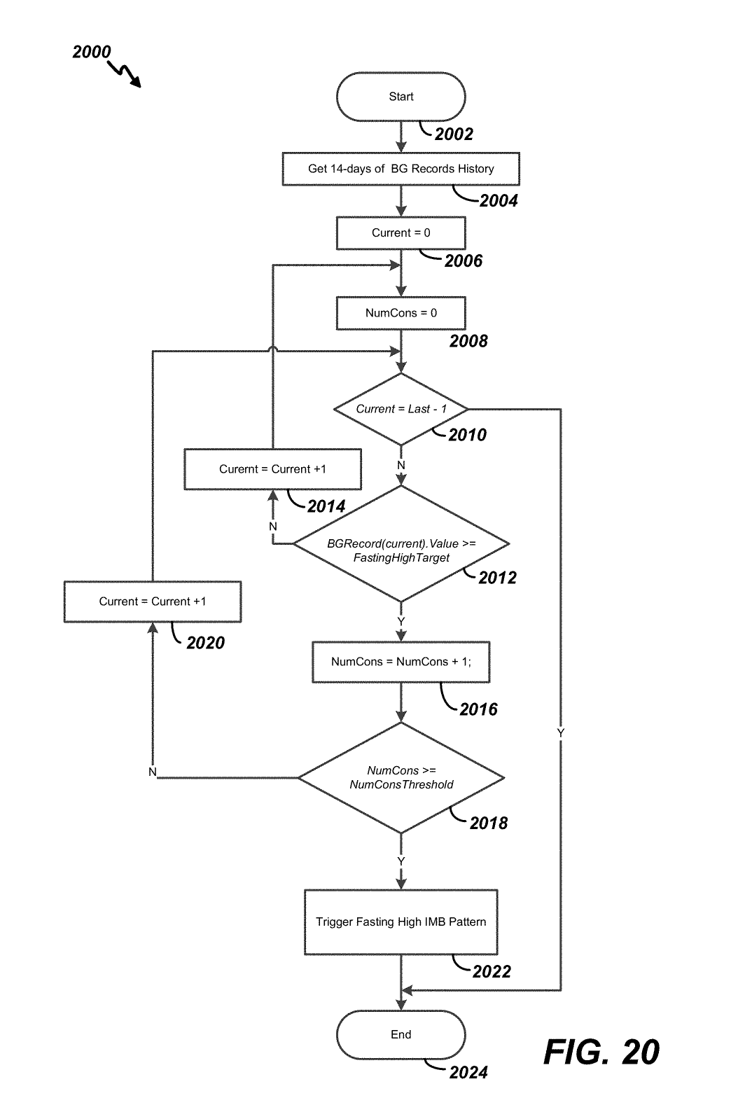

An example of an algorithm or method 2000 for recognizing a fasting high pattern is illustrated as a flowchart in FIG. 20. The method 2000 starts when a new BG record is acquired or a previously acquired record is modified (2002). When a new BG record is acquired or a previously acquired record is modified, the DMS App will trigger this pattern if it detects (within two weeks of data starting from the time stamp of the latest BG Reading) NumConsThreshold or more consecutive BG Readings meal marked as “fasting” that are higher than a predefined parameter FastingTargetHigh. The most recent two weeks of BG readings are retrieved (2004). A BG records index “Current” is initialized to zero (2006) and a counter “NumCons” is initialized to zero (2008). A check is made to determine if the index has reached the last BG record (2010). If so, the method 2000 ends without triggering the pattern (2024). Otherwise, the value of the current BG record is compared to the FastingTargetHigh (2012). If the value of the current BG record is less than the FastingTargetHigh, the index is incremented (2014) and flow returns to resetting counter “NumCons” to zero (2008). Otherwise, NumCons is incremented (2016) and a check to see if NumCons is greater than or equal to NumConsThreshold (2018). If not, the index is incremented (2020) and flow returns to checking to determine if the index has reached the last BG record (2010). Otherwise, the fasting high IMB pattern is triggered (2022) and the method 2000 completes (2024). In other words, the first (most recent) BG reading from the 14-day history with a value above FastingTargetHigh is located. A counter that counts the number of consecutive Fasting High readings is increased by one. The previous BG reading is checked. If the previous BG reading is lower than FastingHighTarget, the counter is reset (NumCons=0), the first next reading (going backwards) higher than FastingTargetHigh is found, and the method 2000 repeats from the beginning. If the previous BG reading is higher than FastingHighTarget, the counter is increased by 1 (NumCons=NumCons+1) and the method 2000 proceeds in the same fashion until the first reading lower than FastingHighTarget is found. Once found, the counter value is checked. If NumCons>=NumConsThreshold, then the pattern is triggered and the Time Range for which this pattern is triggered is identified to the Pattern Manager module 1004. The method 2000 continues from the beginning until the last BG record is reached.

An example of an algorithm or method 2000 for recognizing a fasting high pattern is illustrated as a flowchart in FIG. 20. The method 2000 starts when a new BG record is acquired or a previously acquired record is modified (2002). When a new BG record is acquired or a previously acquired record is modified, the DMS App will trigger this pattern if it detects (within two weeks of data starting from the time stamp of the latest BG Reading) NumConsThreshold or more consecutive BG Readings meal marked as “fasting” that are higher than a predefined parameter FastingTargetHigh. The most recent two weeks of BG readings are retrieved (2004). A BG records index “Current” is initialized to zero (2006) and a counter “NumCons” is initialized to zero (2008). A check is made to determine if the index has reached the last BG record (2010). If so, the method 2000 ends without triggering the pattern (2024). Otherwise, the value of the current BG record is compared to the FastingTargetHigh (2012). If the value of the current BG record is less than the FastingTargetHigh, the index is incremented (2014) and flow returns to resetting counter “NumCons” to zero (2008). Otherwise, NumCons is incremented (2016) and a check to see if NumCons is greater than or equal to NumConsThreshold (2018). If not, the index is incremented (2020) and flow returns to checking to determine if the index has reached the last BG record (2010). Otherwise, the fasting high IMB pattern is triggered (2022) and the method 2000 completes (2024). In other words, the first (most recent) BG reading from the 14-day history with a value above FastingTargetHigh is located. A counter that counts the number of consecutive Fasting High readings is increased by one. The previous BG reading is checked. If the previous BG reading is lower than FastingHighTarget, the counter is reset (NumCons=0), the first next reading (going backwards) higher than FastingTargetHigh is found, and the method 2000 repeats from the beginning. If the previous BG reading is higher than FastingHighTarget, the counter is increased by 1 (NumCons=NumCons+1) and the method 2000 proceeds in the same fashion until the first reading lower than FastingHighTarget is found. Once found, the counter value is checked. If NumCons>=NumConsThreshold, then the pattern is triggered and the Time Range for which this pattern is triggered is identified to the Pattern Manager module 1004. The method 2000 continues from the beginning until the last BG record is reached.

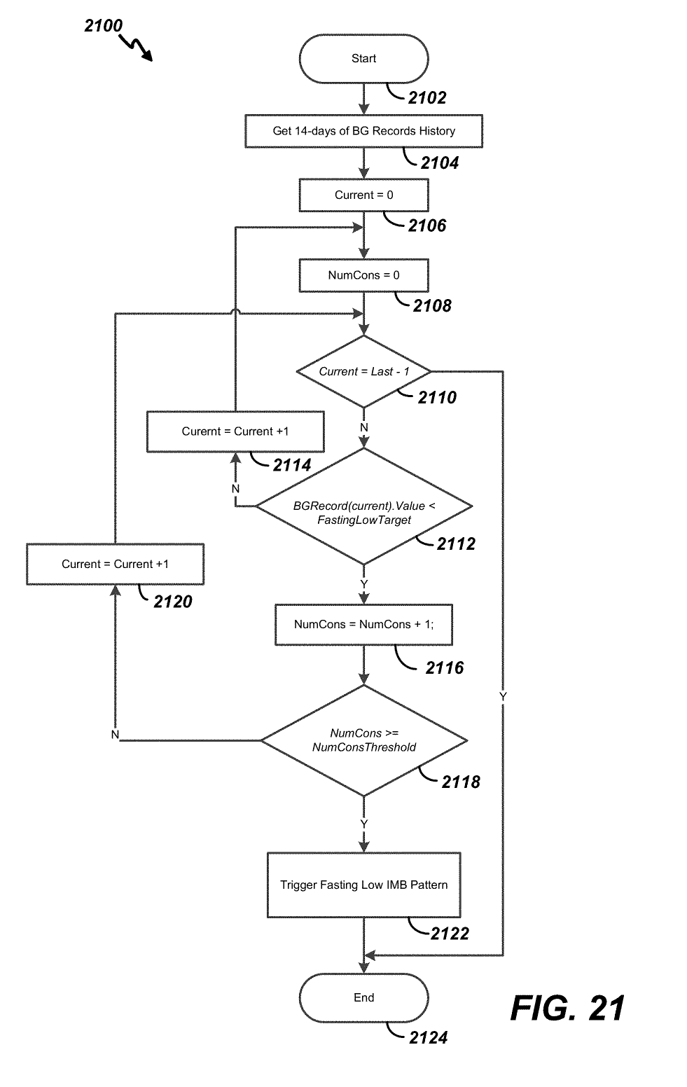

An example of an algorithm or method 2100 for recognizing a fasting low pattern is illustrated as a flowchart in FIG. 21. The method 2100 starts when a new BG record is acquired or a previously acquired record is modified (2102). When a new BG record is acquired or a previously acquired record is modified, the DMS App will trigger this pattern if it detects (within two weeks of data starting from the time stamp of the latest BG Reading) NumConsThreshold or more consecutive BG Readings meal marked as “fasting” that are lower than a predefined parameter FastingTargetLow. The most recent two weeks of BG readings are retrieved (2104). A BG records index “Current” is initialized to zero (2106) and a counter “NumCons” is initialized to zero (2108). A check is made to determine if the index has reached the last BG record (2110). If so, the method 2100 ends without triggering the pattern (2124). Otherwise, the value of the current BG record is compared to the FastingTargetLow (2112). If the value of the current BG record is greater than the FastingTargetLow, the index is incremented (2114) and flow returns to resetting counter “NumCons” to zero (2108). Otherwise, NumCons is incremented (2116) and a check to see if NumCons is greater than or equal to NumConsThreshold (2118). If not, the index is incremented (2120) and flow returns to checking to determine if the index has reached the last BG record (2110). Otherwise (i.e., NumCons is greater than or equal to NumConsThreshold), the fasting low IMB pattern is triggered (2122) and the method 2100 completes (2124). In other words, the first (most recent) BG reading from the 14-day history with a value below FastingTargetLow is located. A counter that counts the number of consecutive Fasting Low readings is increased by one. The previous BG reading is checked. If the previous BG reading is higher than FastingLowTarget, the counter is reset (NumCons=0), the first next reading (going backwards) lower than FastingTargetLow is found, and the method 2100 repeats from the beginning. If the previous BG reading is lower than FastingLowTarget, the counter is increased by 1 (NumCons=NumCons+1) and the method 2100 proceeds in the same fashion until the first reading higher than FastingLowTarget is found. Once found, the counter value is checked. If NumCons>=NumConsThreshold, then the pattern is triggered and the Time Range for which this pattern is triggered is identified to the Pattern Manager module 1004. The method 2100 continues from the beginning until the last BG record is reached.

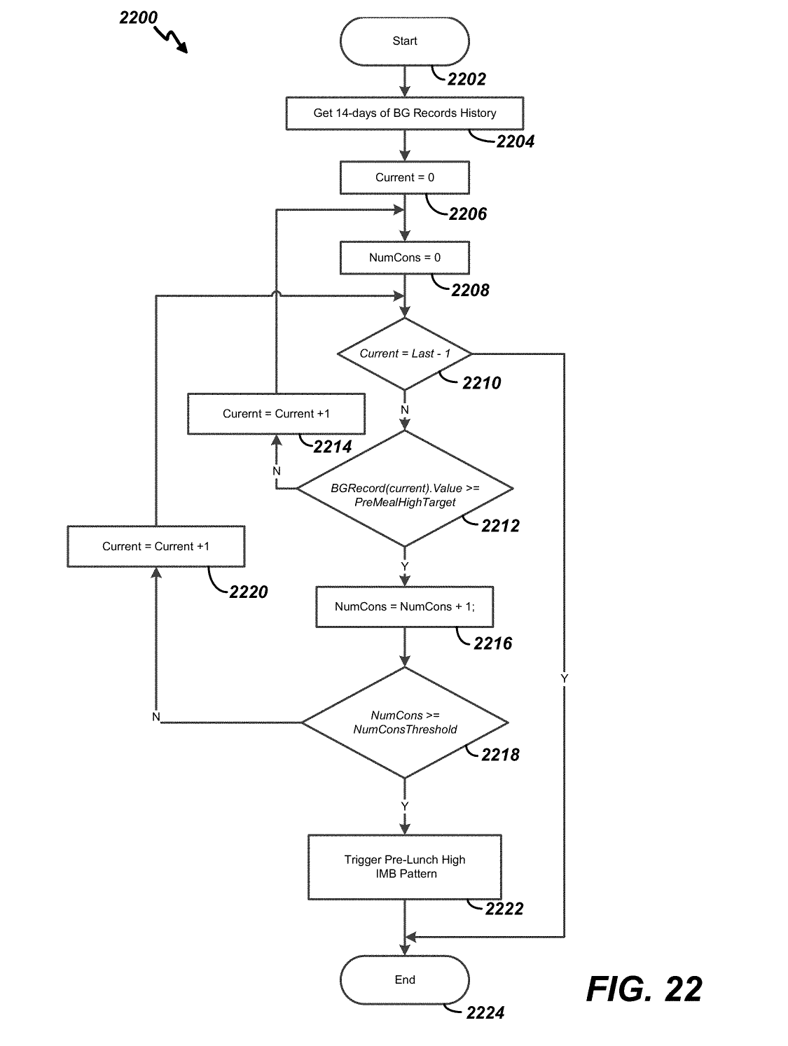

An example of an algorithm or method 2200 for recognizing a pre-lunch high pattern is illustrated as a flowchart in FIG. 22. The method 2200 starts when a new BG record is acquired or a previously acquired record is modified (2202). When a new BG record is acquired or a previously acquired record is modified, the DMS App will trigger this pattern if it detects (within two weeks of data starting from the time stamp of the latest BG Reading) NumConsThreshold (e.g., 3) or more consecutive BG Readings that occurred within the Lunch Day Divider and meal marked as “before meal” that are higher than a predefined parameter PreMealHighTarget. The most recent two weeks of BG readings are retrieved (2204). A BG records index “Current” is initialized to zero (2206) and a counter “NumCons” is initialized to zero (2208). A check is made to determine if the index has reached the last BG record (2210). If so, the method 2200 ends without triggering the pattern (2224). Otherwise, the value of the current BG record is compared to the PreMealHighTarget (2212). If the value of the current BG record is less than the PreMealHighTarget, the index is incremented (2214) and flow returns to resetting counter “NumCons” to zero (2208). Otherwise, NumCons is incremented (2216) and a check to see if NumCons is greater than or equal to NumConsThreshold (2218). If not, the index is incremented (2220) and flow returns to checking to determine if the index has reached the last BG record (2210). Otherwise (i.e., NumCons is greater than or equal to NumConsThreshold), the pre-lunch high IMB pattern is triggered (2222) and the method 2200 completes (2224). In other words, the first (most recent) BG reading from the 14-day history with a value above PreMealHighTarget is located. A counter that counts the number of consecutive pre-lunch high readings is increased by one. The previous BG reading is checked. If the previous BG reading is lower than PreMealHighTarget, the counter is reset (NumCons=0), the first next reading (going backwards) higher than PreMealHighTarget is found, and the method 2200 repeats from the beginning. If the previous BG reading is higher than PreMealHighTarget, the counter is increased by 1 (NumCons=NumCons+1) and the method 2200 proceeds in the same fashion until the first reading lower than PreMealHighTarget is found. Once found, the counter value is checked. If NumCons>=NumConsThreshold, then the pattern is triggered and the Time Range for which this pattern is triggered is identified to the Pattern Manager module 1004. The method 2200 continues from the beginning until the last BG record is reached.

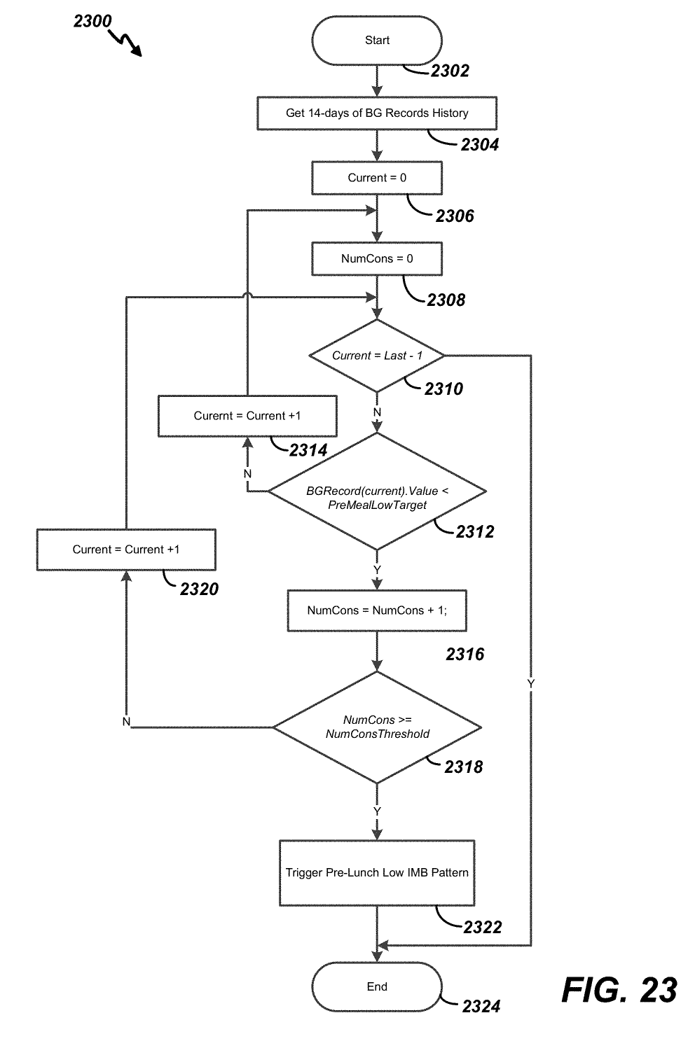

An example of an algorithm or method 2300 for recognizing a pre-lunch low pattern is illustrated as a flowchart in FIG. 23. The method 2300 starts when a new BG record is acquired or a previously acquired record is modified (2302). When a new BG record is acquired or a previously acquired record is modified, the DMS App will trigger this pattern if it detects (within two weeks of data starting from the time stamp of the latest BG Reading) NumConsThreshold or more consecutive BG Readings that occurred within the Lunch Day Divider and meal marked as “before meal” that are lower than a predefined parameter PreMealLowTarget. The most recent two weeks of BG readings are retrieved (2304). A BG records index “Current” is initialized to zero (2306) and a counter “NumCons” is initialized to zero (2308). A check is made to determine if the index has reached the last BG record (2310). If so, the method 2300 ends without triggering the pattern (2324). Otherwise, the value of the current BG record is compared to the PreMealLowTarget (2312). If the value of the current BG record is greater than the PreMealLowTarget, the index is incremented (2314) and flow returns to resetting counter “NumCons” to zero (2308). Otherwise, NumCons is incremented (2316) and a check to see if NumCons is greater than or equal to NumConsThreshold (2318). If not, the index is incremented (2320) and flow returns to checking to determine if the index has reached the last BG record (2310). Otherwise (i.e., NumCons is greater than or equal to NumConsThreshold), the pre-lunch low IMB pattern is triggered (2322) and the method 2300 completes (2324). In other words, the first (most recent) BG reading from the 14-day history with a value below PreMealLowTarget is located. A counter that counts the number of consecutive Pre-Lunch Low readings is increased by one. The previous BG reading is checked. If the previous BG reading is higher than PreMealLowTarget, the counter is reset (NumCons=0), the first next reading (going backwards) lower than PreMealLowTarget is found, and the method 2300 repeats from the beginning. If the previous BG reading is lower than PreMealLowTarget, the counter is increased by 1 (NumCons=NumCons+1) and the method 2300 proceeds in the same fashion until the first reading higher than PreMealLowTarget is found. Once found, the counter value is checked. If NumCons>=NumConsThreshold, then the pattern is triggered and the Time Range for which this pattern is triggered is identified to the Pattern Manager module 1004. The method 2300 continues from the beginning until the last BG record is reached.

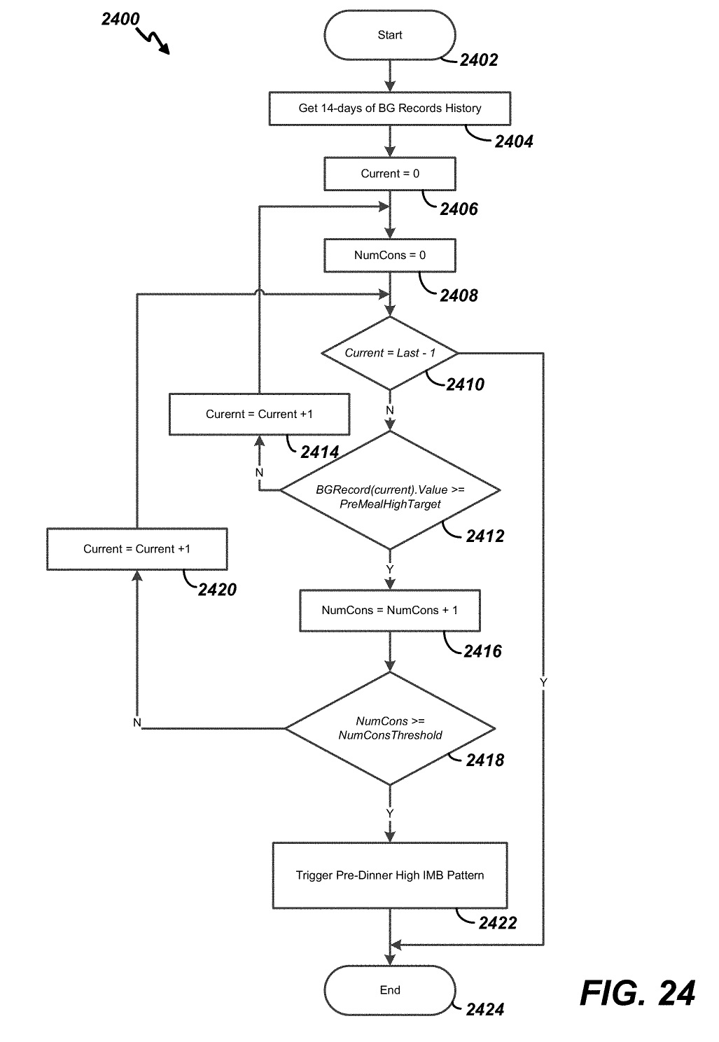

An example of an algorithm or method 2400 for recognizing a pre-dinner high pattern is illustrated as a flowchart in FIG. 24. The method 2400 starts when a new BG record is acquired or a previously acquired record is modified (2402). When a new BG record is acquired or a previously acquired record is modified, the DMS App will trigger this pattern if it detects (within two weeks of data starting from the time stamp of the latest BG Reading) NumConsThreshold (e.g., 3) or more consecutive BG Readings that occurred within the Dinner Day Divider and meal marked as “before meal” that are higher than a predefined parameter PreMealHighTarget. The most recent two weeks of BG readings are retrieved (2404). A BG records index “Current” is initialized to zero (2406) and a counter “NumCons” is initialized to zero (2408). A check is made to determine if the index has reached the last BG record (2410). If so, the method 2400 ends without triggering the pattern (2424). Otherwise, the value of the current BG record is compared to the PreMealHighTarget (2412). If the value of the current BG record is less than the PreMealHighTarget, the index is incremented (2414) and flow returns to resetting counter “NumCons” to zero (2408). Otherwise, NumCons is incremented (2416) and a check to see if NumCons is greater than or equal to NumConsThreshold (2418). If not, the index is incremented (2420) and flow returns to checking to determine if the index has reached the last BG record (2410). Otherwise (i.e., NumCons is greater than or equal to NumConsThreshold), the pre-dinner high IMB pattern is triggered (2422) and the method 2400 completes (2424). In other words, the first (most recent) BG reading from the 14-day history with a value above PreMealHighTarget is located. A counter that counts the number of consecutive pre-dinner high readings is increased by one. The previous BG reading is checked. If the previous BG reading is lower than PreMealHighTarget, the counter is reset (NumCons=0), the first next reading (going backwards) higher than PreMealHighTarget is found, and the method 2400 repeats from the beginning. If the previous BG reading is higher than PreMealHighTarget, the counter is increased by 1 (NumCons=NumCons+1) and the method 2400 proceeds in the same fashion until the first reading lower than PreMealHighTarget is found. Once found, the counter value is checked. If NumCons>=NumConsThreshold, then the pattern is triggered and the Time Range for which this pattern is triggered is identified to the Pattern Manager module 1004. The method 2400 continues from the beginning until the last BG record is reached.

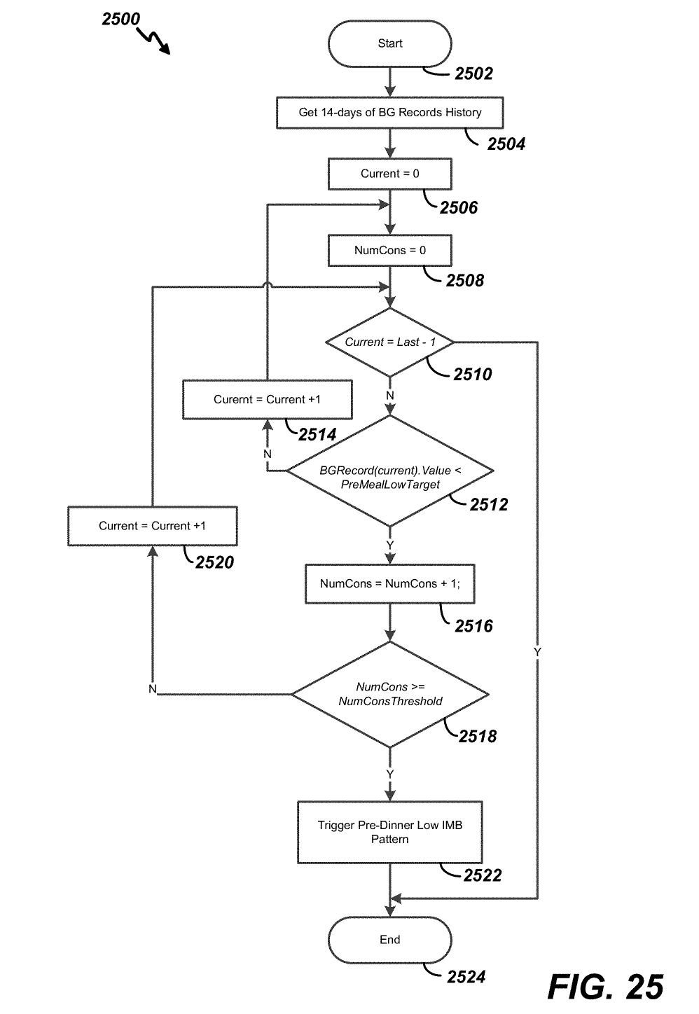

An example of an algorithm or method 2500 for recognizing a pre-dinner low pattern is illustrated as a flowchart in FIG. 25. The method 2500 starts when a new BG record is acquired or a previously acquired record is modified (2502). When a new BG record is acquired or a previously acquired record is modified, the DMS App will trigger this pattern if it detects (within two weeks of data starting from the time stamp of the latest BG Reading) NumConsThreshold or more consecutive BG Readings that occurred within the Dinner Day Divider and meal marked as “before meal” that are lower than a predefined parameter PreMealLowTarget. The most recent two weeks of BG readings are retrieved (2504). A BG records index “Current” is initialized to zero (2506) and a counter “NumCons” is initialized to zero (2508). A check is made to determine if the index has reached the last BG record (2510). If so, the method 2500 ends without triggering the pattern (2524). Otherwise, the value of the current BG record is compared to the PreMealLowTarget (2512). If the value of the current BG record is greater than the PreMealLowTarget, the index is incremented (2514) and flow returns to resetting counter “NumCons” to zero (2508). Otherwise, NumCons is incremented (2516) and a check to see if NumCons is greater than or equal to NumConsThreshold (2518). If not, the index is incremented (2520) and flow returns to checking to determine if the index has reached the last BG record (2510). Otherwise (i.e., NumCons is greater than or equal to NumConsThreshold), the pre-dinner low IMB pattern is triggered (2522) and the method 2500 completes (2524). In other words, the first (most recent) BG reading from the 14-day history with a value below PreMealLowTarget is located. A counter that counts the number of consecutive Pre-Dinner Low readings is increased by one. The previous BG reading is checked. If the previous BG reading is higher than PreMealLowTarget, the counter is reset (NumCons=0), the first next reading (going backwards) lower than PreMealLowTarget is found, and the method 2500 repeats from the beginning. If the previous BG reading is lower than PreMealLowTarget, the counter is increased by 1 (NumCons=NumCons+1) and the method 2500 proceeds in the same fashion until the first reading higher than PreMealLowTarget is found. Once found, the counter value is checked. If NumCons>=NumConsThreshold, then the pattern is triggered and the Time Range for which this pattern is triggered is identified to the Pattern Manager module 1004. The method 2500 continues from the beginning until the last BG record is reached.

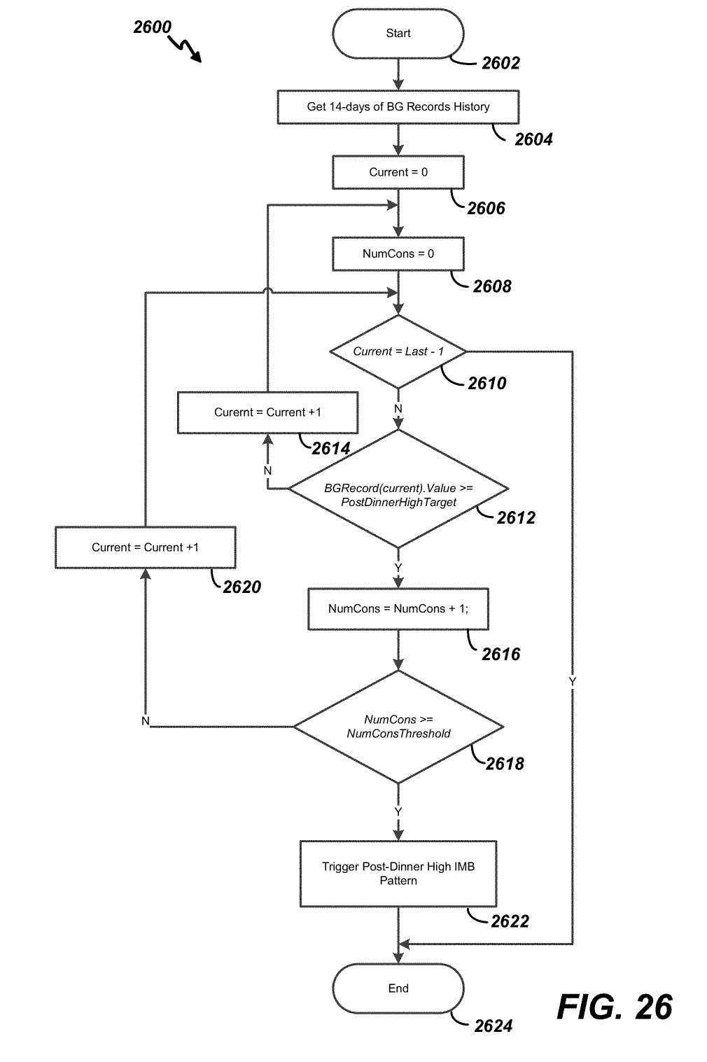

An example of an algorithm or method 2600 for recognizing a post-dinner high pattern is illustrated as a flowchart in FIG. 26. The method 2600 starts when a new BG record is acquired or a previously acquired record is modified (2602). When a new BG record is acquired or a previously acquired record is modified, the DMS App will trigger this pattern if it detects (within two weeks of data starting from the time stamp of the latest BG Reading) NumConsThreshold (e.g., 3) or more consecutive BG Readings that occurred within the Dinner Day Divider and meal marked as “before meal” that are higher than a predefined parameter PostMealHighTarget. The most recent two weeks of BG readings are retrieved (2604). A BG records index “Current” is initialized to zero (2606) and a counter “NumCons” is initialized to zero (2608). A check is made to determine if the index has reached the last BG record (2610). If so, the method 2600 ends without triggering the pattern (2624). Otherwise, the value of the current BG record is compared to the PostMealHighTarget (2612). If the value of the current BG record is less than the PostMealHighTarget, the index is incremented (2614) and flow returns to resetting counter “NumCons” to zero (2608). Otherwise, NumCons is incremented (2616) and a check to see if NumCons is greater than or equal to NumConsThreshold (2618). If not, the index is incremented (2620) and flow returns to checking to determine if the index has reached the last BG record (2610). Otherwise (i.e., NumCons is greater than or equal to NumConsThreshold), the post-dinner high IMB pattern is triggered (2622) and the method 2600 completes (2624). In other words, the first (most recent) BG reading from the 14-day history with a value above PostMealHighTarget is located. A counter that counts the number of consecutive post-dinner high readings is increased by one. The previous BG reading is checked. If the previous BG reading is lower than PostMealHighTarget, the counter is reset (NumCons=0), the first next reading (going backwards) higher than PostMealHighTarget is found, and the method 2600 repeats from the beginning. If the previous BG reading is higher than PostMealHighTarget, the counter is increased by 1 (NumCons=NumCons+1) and the method 2600 proceeds in the same fashion until the first reading lower than PostMealHighTarget is found. Once found, the counter value is checked. If NumCons>=NumConsThreshold, then the pattern is triggered and the Time Range for which this pattern is triggered is identified to the Pattern Manager module 1004. The method 2600 continues from the beginning until the last BG record is reached.

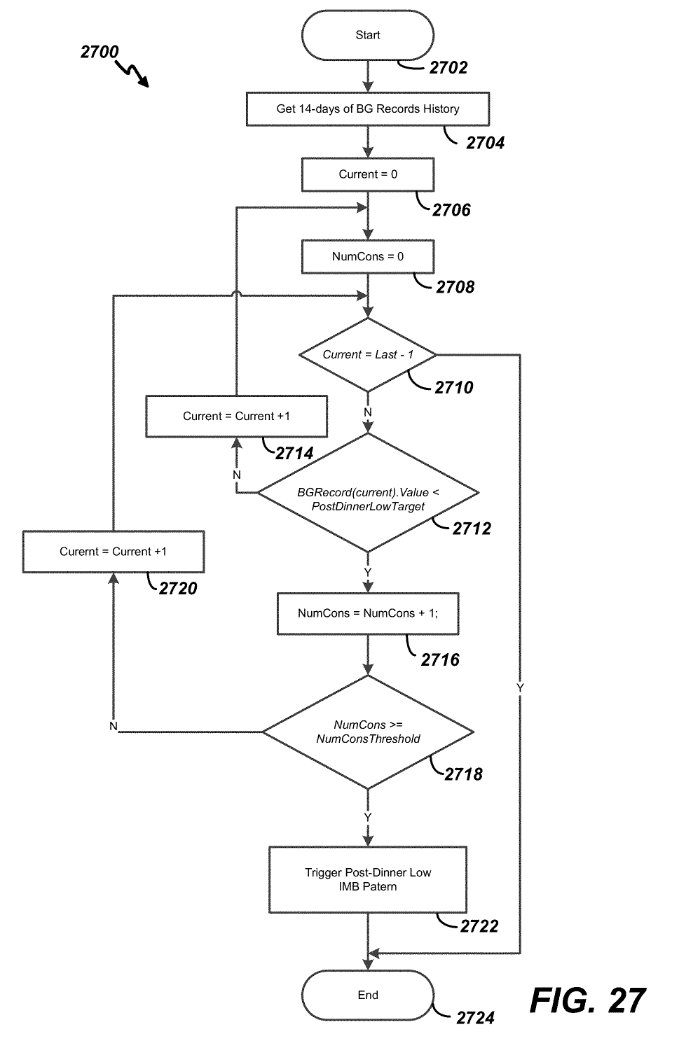

An example of an algorithm or method 2700 for recognizing a post-dinner low pattern is illustrated as a flowchart in FIG. 27. The method 2700 starts when a new BG record is acquired or a previously acquired record is modified (2702). When a new BG record is acquired or a previously acquired record is modified, the DMS App will trigger this pattern if it detects (within two weeks of data starting from the time stamp of the latest BG Reading) NumConsThreshold or more consecutive BG Readings that occurred within the Dinner Day Divider and meal marked as “before meal” that are lower than a predefined parameter PostMealLowTarget. The most recent two weeks of BG readings are retrieved (2704). A BG records index “Current” is initialized to zero (2706) and a counter “NumCons” is initialized to zero (2708). A check is made to determine if the index has reached the last BG record (2710). If so, the method 2700 ends without triggering the pattern (2724). Otherwise, the value of the current BG record is compared to the PostMealLowTarget (2712). If the value of the current BG record is greater than the PostMealLowTarget, the index is incremented (2714) and flow returns to resetting counter “NumCons” to zero (2708). Otherwise, NumCons is incremented (2716) and a check to see if NumCons is greater than or equal to NumConsThreshold (2718). If not, the index is incremented (2720) and flow returns to checking to determine if the index has reached the last BG record (2710). Otherwise (i.e., NumCons is greater than or equal to NumConsThreshold), the post-dinner low IMB pattern is triggered (2722) and the method 2700 completes (2724). In other words, the first (most recent) BG reading from the 14-day history with a value below PostMealLowTarget is located. A counter that counts the number of consecutive Post-Dinner Low readings is increased by one. The previous BG reading is checked. If the previous BG reading is higher than PostMealLowTarget, the counter is reset (NumCons=0), the first next reading (going backwards) lower than PostMealLowTarget is found, and the method 2700 repeats from the beginning. If the previous BG reading is lower than PostMealLowTarget, the counter is increased by 1 (NumCons=NumCons+1) and the method 2700 proceeds in the same fashion until the first reading higher than PostMealLowTarget is found. Once found, the counter value is checked. If NumCons>=NumConsThreshold, then the pattern is triggered and the Time Range for which this pattern is triggered is identified to the Pattern Manager module 1004. The method 2700 continues from the beginning until the last BG record is reached.

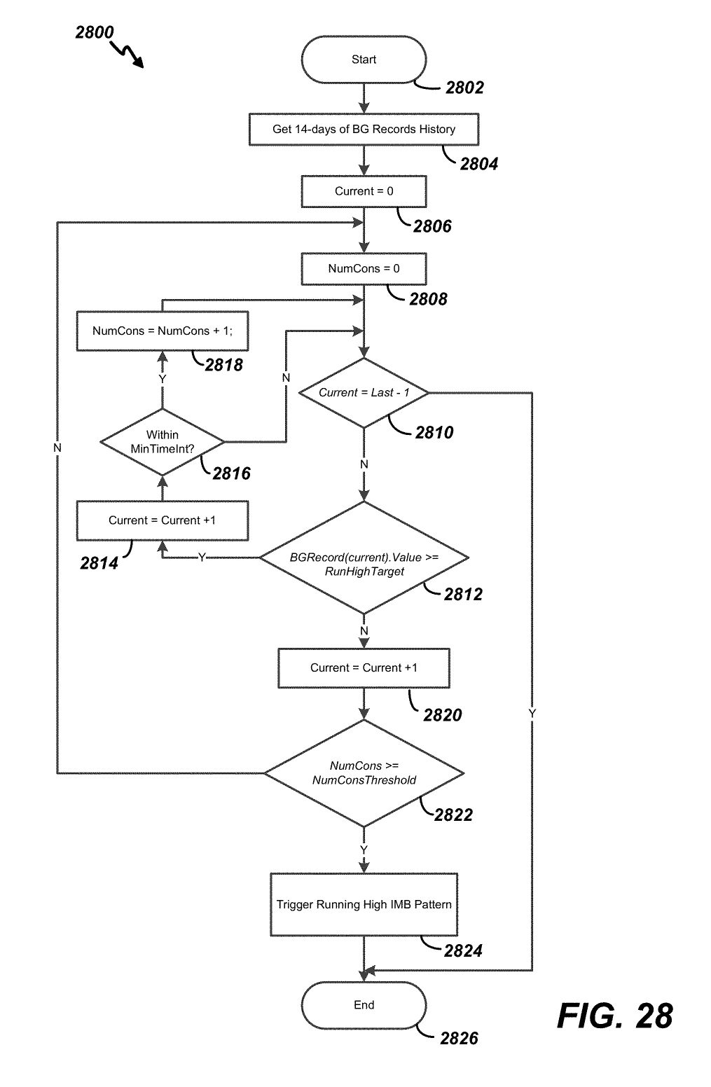

An example of an algorithm or method 2800 for recognizing a running high pattern is illustrated as a flowchart in FIG. 28. The method 2800 starts when a new BG record is acquired or a previously acquired record is modified (2802). When a new BG record is acquired or a previously acquired record is modified, the DMS App will trigger this pattern if it detects (within two weeks of data starting from the time stamp of the latest BG Reading) NumConsThreshold (e.g., 3) or more consecutive BG Readings that occurred within MinTimeInterval (e.g., a minimum time interval between two consecutive BG measurements in order for them not to be considered correlated) apart from each other and are higher than a predefined parameter RunHighTarget. The most recent two weeks of BG readings are retrieved (2804). A BG records index “Current” is initialized to zero (2806) and a counter “NumCons” is initialized to zero (2808). A check is made to determine if the index has reached the last BG record (2810). If so, the method 2800 ends without triggering the pattern (2826). Otherwise, the value of the current BG record is compared to the RunHighTarget (2812). If the value of the current BG record is greater than or equal to the RunHighTarget, the index (Current) is incremented (2814). A check is then made to determine if the current BG record occurred within the MinTimeInterval of the previous BG record (2816). If not, flow returns to checking if the index has reached the last BG record (2810). Otherwise, the counter “NumCons” is incremented (2818) and flow returns to checking if the index has reached the last BG record (2810). If the value of the current BG record is less than the RunHighTarget, the index (Current) is incremented (2820) and then a check is made to see if NumCons is greater than or equal to NumConsThreshold (2822). If not, flow returns to resetting NumCons to zero (2808). Otherwise (i.e., NumCons is greater than or equal to NumConsThreshold), the running high IMB pattern is triggered (2824) and the method 2800 completes (2826). In other words, the first (most recent) BG reading from the 14-day history with a value above RunHighTarget is located. A counter that counts the number of consecutive high readings is increased by one. The previous BG reading is checked. If the previous BG reading is lower than RunHighTarget, the counter is reset (NumCons=0), the first next reading (going backwards) higher than RunHighTarget is found, and the method 2800 repeats from the beginning. If the previous BG reading is higher than RunHighTarget and if the time between the current reading and the previous reading is less than MinTimeInterval, the method 2800 proceeds in the same fashion until the first reading lower than RunHighTarget is found. Once found, the counter value is checked. If NumCons>=NumConsThreshold, then the pattern is triggered and the Time Range for which this pattern is triggered is identified to the Pattern Manager module 1004. The method 2800 continues from the beginning until the last BG record is reached. If the previous BG reading is higher than RunHighTarget and if the time between the current reading and the previous reading is greater than or equal to the MinTimeInterval, the counter is increased by 1 (NumCons=NumCons+1) and the method 2800 proceeds in the same fashion until the first reading lower than RunHighTarget is found. Once found, the counter value is checked. If NumCons>=NumConsThreshold, then the pattern is triggered and the Time Range for which this pattern is triggered is identified to the Pattern Manager module 1004. The method 2800 continues from the beginning until the last BG record is reached.

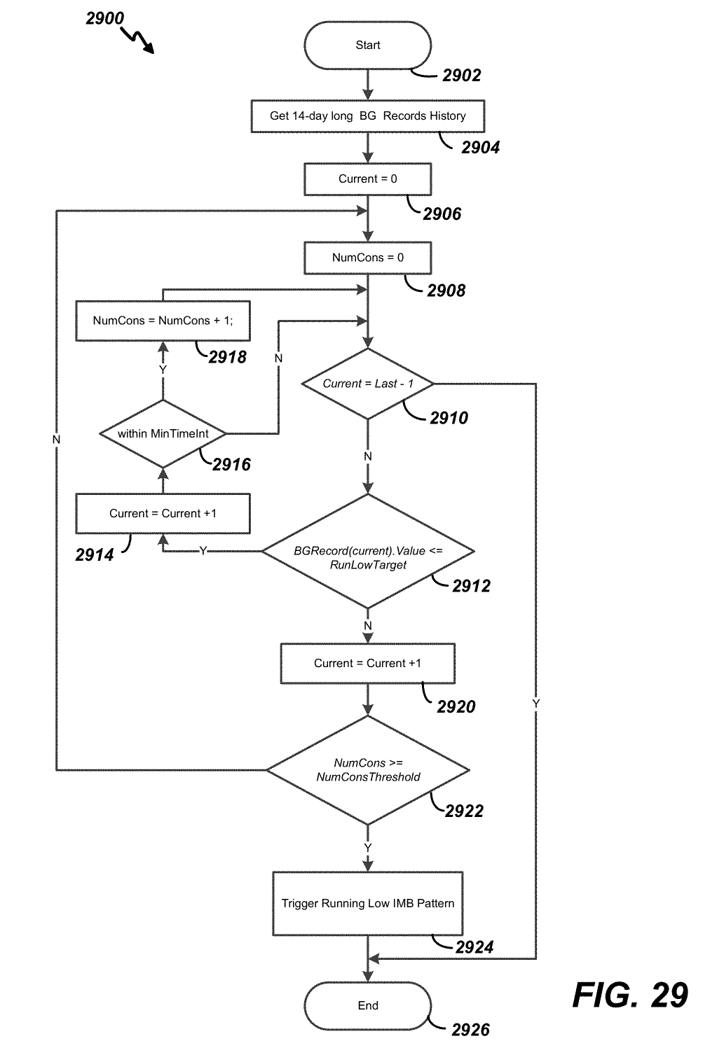

An example of an algorithm or method 2900 for recognizing a running low pattern is illustrated as a flowchart in FIG. 29. The method 2900 starts when a new BG record is acquired or a previously acquired record is modified (2902). When a new BG record is acquired or a previously acquired record is modified, the DMS App will trigger this pattern if it detects (within two weeks of data starting from the time stamp of the latest BG Reading) NumConsThreshold (e.g., 3) or more consecutive BG Readings that occurred within MinTimeInterval (e.g., a minimum time interval between two consecutive BG measurements in order for them not to be considered correlated) apart from each other and are lower than a predefined parameter RunLowTarget. The most recent two weeks of BG readings are retrieved (2904). A BG records index “Current” is initialized to zero (2906) and a counter “NumCons” is initialized to zero (2908). A check is made to determine if the index has reached the last BG record (2910). If so, the method 2900 ends without triggering the pattern (2926). Otherwise, the value of the current BG record is compared to the RunLowTarget (2912). If the value of the current BG record is less than or equal to the RunLowTarget, the index (Current) is incremented (2914). A check is then made to determine if the current BG record occurred within the MinTimeInterval of the previous BG record (2916). If not, flow returns to checking if the index has reached the last BG record (2910). Otherwise, the counter “NumCons” is incremented (2918) and then flow returns to checking if the index has reached the last BG record (2910). If the value of the current BG record is greater than the RunLowTarget, the index (Current) is incremented (2920) and then a check is made to see if NumCons is greater than or equal to NumConsThreshold (2922). If not, flow returns to resetting NumCons to zero (2908). Otherwise (i.e., NumCons is greater than or equal to NumConsThreshold), the running low IMB pattern is triggered (2924) and the method 2900 completes (2926). In other words, the first (most recent) BG reading from the 14-day history with a value below RunLowTarget is located. A counter that counts the number of consecutive low readings is increased by one. The previous BG reading is checked. If the previous BG reading is higher than RunLowTarget, the counter is reset (NumCons=0), the first next reading (going backwards) higher than RunLowTarget is found, and the method 2900 repeats from the beginning. If the previous BG reading is lower than RunLowTarget and if the time between the current reading and the previous reading is less than MinTimeInterval, the method 2900 proceeds in the same fashion until the first reading higher than RunLowTarget is found. Once found, the counter value is checked. If NumCons>=NumConsThreshold, then the pattern is triggered and the Time Range for which this pattern is triggered is identified to the Pattern Manager module 1004. The method 2900 continues from the beginning until the last BG record is reached. If the previous BG reading is lower than RunLowTarget and if the time between the current reading and the previous reading is greater than or equal to the MinTimeInterval, the counter is increased by 1 (NumCons=NumCons+1) and the method 2900 proceeds in the same fashion until the first reading higher than RunLowTarget is found. Once found, the counter value is checked. If NumCons>=NumConsThreshold, then the pattern is triggered and the Time Range for which this pattern is triggered is identified to the Pattern Manager module 1004. The method 2900 continues from the beginning until the last BG record is reached.

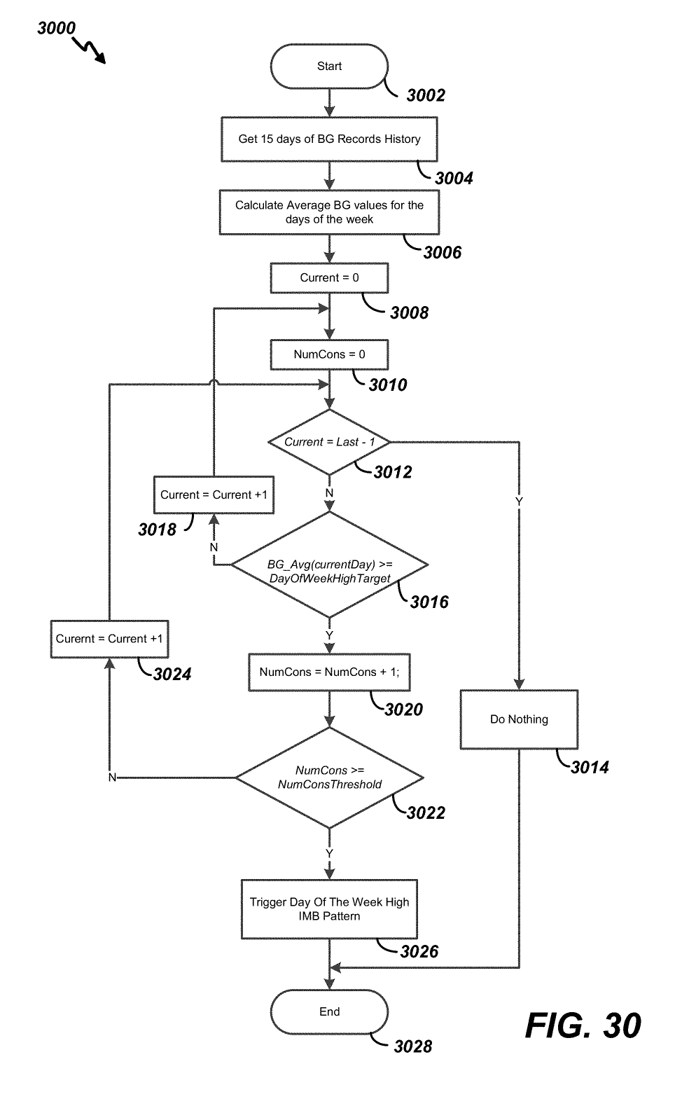

An example of an algorithm or method 3000 for recognizing a day of the week high pattern is illustrated as a flowchart in FIG. 30. The method 30000 starts when a new BG record is acquired or a previously acquired record is modified (3002). When a new BG record is acquired or a previously acquired record is modified, the DMS App will trigger this pattern if the App detects (within three weeks of data extending back in time from the time stamp of the latest BG Reading) that average values of BG records for NumConsThreshold consecutive days of the week were higher than DayOfWeekHighTarget (e.g., 110% of After Meal/No Mark High). Note that for this pattern, data from fifteen days (spanning three weeks) to 21 days of BG records history is used in order for this pattern to be triggered. Further note, assume that daily averages for each day of the week for the past 15 days have already been computed and stored. When a new reading or readings come in, new (in the case of new readings spreading over multiple days) daily averages are computed or just the last one is updated (in the case of new readings influencing only one last day of history) and a check whether there are three or more consecutive days of the week with an average BG value higher than DayOfWeekHighTarget (e.g., “3 or more consecutive Fridays in a row”). The most recent 15 days to three weeks of BG readings are retrieved (3004). Average BG values are calculated for each of the days of the week (AVGday) (3006). A BG records index “Current” is initialized to zero (3008) and a counter “NumCons” is initialized to zero (3010). A check is made to determine if the index has reached the last BG record (3012). If so, the pattern is not triggered (3014) and the method 3000 ends (3028). Otherwise, the value of the average for the current day of the week is compared to the DayOfWeekHighTarget (3016). If the value of the average for the current day of the week is less than the DayOfWeekHighTarget, the counter “Current” is incremented (3018) and flow returns to resetting counter “NumCons” to zero (3010). Otherwise, NumCons is incremented (3020) and then a check if NumComs is greater than or equal to NumConsThreshold (3022). If not, “Current” is incremented (3024) and flow returns to checking to determine if the index has reached the last BG record (3012). Otherwise (NumComs is greater than or equal to NumConsThreshold), the day of the week high IMB pattern is triggered (3026) and the method 3000 ends (3028).

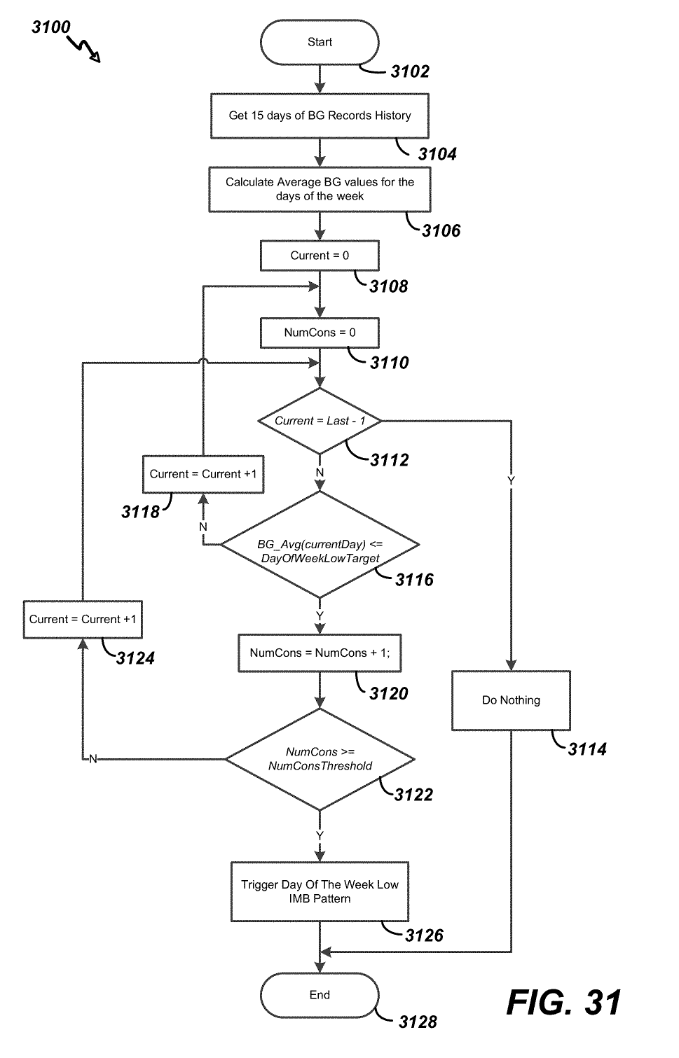

An example of an algorithm or method 3100 for recognizing a day of the week low pattern is illustrated as a flowchart in FIG. 31. The method 31000 starts when a new BG record is acquired or a previously acquired record is modified (3102). When a new BG record is acquired or a previously acquired record is modified, the DMS App will trigger this pattern if the App detects (within three weeks of data extending back in time from the time stamp of the latest BG Reading) that average values of BG records for NumConsThreshold consecutive days of the week were lower than DayOfWeekLowTarget (e.g., 80% of Overall Low Value). Note that for this pattern, data from fifteen days (spanning three weeks) to 21 days of BG records history is used in order for this pattern to be triggered. Further note, assume that daily averages for each day of the week for the past 15 days have already been computed and stored. When a new reading or readings come in, new (in the case of new readings spreading over multiple days) daily averages are computed or just the last one is updated (in the case of new readings influencing only one last day of history) and a check whether there are three or more consecutive days of the week with an average BG value lower than DayOfWeekLowTarget (e.g., “3 or more consecutive Mondays in a row”). The most recent 15 days to three weeks of BG readings are retrieved (3104). Average BG values are calculated for each of the days of the week (AVGday) (3106). A BG records index “Current” is initialized to zero (3108) and a counter “NumCons” is initialized to zero (3110). A check is made to determine if the index has reached the last BG record (3112). If so, the pattern is not triggered (3114) and the method 3100 ends (3128). Otherwise, the value of the average for the current day of the week is compared to the DayOfWeekLowTarget (3116). If the value of the average for the current day of the week is greater than the DayOfWeekLowTarget, the counter “Current” is incremented (3118) and flow returns to resetting counter “NumCons” to zero (3110). Otherwise, NumCons is incremented (3120) and then a check if NumComs is greater than or equal to NumConsThreshold (3122). If not, “Current” is incremented (3124) and flow returns to checking to determine if the index has reached the last BG record (3112). Otherwise (NumComs is greater than or equal to NumConsThreshold), the day of the week low IMB pattern is triggered (3126) and the method 3100 ends (3128).

Within the IMB Manager 704, the Pattern Manager module 1004 is responsible for calling the above-described IMB algorithms (via the IMB Algorithm Execution sub-module 1010), processing detected patterns, creating and scheduling notifications for Identified patterns, scheduling execution of IMB Pattern Maps (e.g., related user interface displays and interactive menus), enabling the user to set up pattern goals, initiating and dismissing constraints and filters (e.g., Band/Testing Frequency Goal/Quarantine) for pattern notifications, following up with user's progress on managing patterns through notifications and reminders, providing informational, motivational and behavioral messages and recording user's notes about associated triggered patterns and BG Records (if applicable) related to the associated patterns.

The Pattern Manager module 1004 communicates that a pattern has been newly detected to the user in different ways. A notification of the specific pattern detection followed by corresponding IMB Pattern Map (depending on user's decision) can be presented to the user. If there is more than one IMB pattern detected, the DMS App can limit the notification to a single notice that includes multiple names of the detected patterns in a prioritized list for the user to easily review the patterns. In addition, the user can access detected patterns via a Patterns Viewer that presents the patterns in an organized and prioritized manner. If the DMS App is active, the Pattern Manager will take over the screen of the Smart Device and present a “Pattern Detection Screen”—notification of the detected pattern within which the user is provided an option to go through the Pattern Map Flow and be guided through details and explanations of the detected patterns.

Operation of the Pattern Manager is based on the following parameters which define each of the patterns: Band, Type, Rank, Quarantine (Embargo) Period, Incubation Period, and Minimal Time Spacing. The Band parameter represents the level of “Pattern Feedback” where the user can choose not to be notified about all the patterns that the Pattern Manager may have detected. In other words, even if the Pattern Manager detected a certain pattern, this event will not be communicated to the user as a New Pattern Detected Notification if the Band the user selected does not include that particular pattern (e.g., an “Out-of-band” pattern) as is the case with the “In-band” patterns.

The following table provides an example arrangement for organizing the patterns into bands. The user can choose which bands of patterns for which to receive notification.

| # | Pattern Name | Band |
|---|---|---|
| 1 | Critical Low/Meter LO | 1 |
| 2 | Critical High/Meter HI | 1 |
| 3 | Testing Frequency Low (No Goal) | 2 |
| | Testing Frequency Low (Goal set) | |
| 4 | Testing Frequency Fair (No Goal Set) | 2 |
| | Testing Frequency Fair (Goal Set) | |
| 5 | Testing Frequency Good (No Goal Set) | 2 |
| | Testing Frequency Good (Goal Set) | |
| 6 | Testing Mostly Same Time | 2 |
| 7 | High Time of Day | 6 |
| 8 | Low Time of Day | 6 |
| 9 | Best Time of Day | 6 |
| 10 | Fasting High | 4 |
| 11 | Fasting Low | 4 |
| 12 | Pre-Lunch High | 4 |
| 13 | Pre-Lunch Low | 4 |
| 14 | Pre-Dinner High | 4 |
| 15 | Pre-Dinner Low | 4 |
| 16 | Post-Dinner High | 4 |
| 17 | Post-Dinner Low | 4 |
| 18 | Running High | 3 |
| 19 | Running Low | 3 |
| 20 | Day of the Week High (DOW High) | 5 |
| 21 | Day of the Week Low (DOW Low) | 5 |

The Type parameter represents a grouping of all the patterns based on the type of the events that trigger the pattern. This is relevant when multiple patterns are detected at the same time. For example, consistent high glucose is evaluated in a number of different ways. This allows the DMS to avoid bombarding the user with seemingly redundant separate alerts for patterns of similar nature.

The Rank parameter represents the priority level of a particular pattern. It is assigned to each pattern in the group (Type) and it is unique for that particular pattern within the Type (in some embodiments, no two patterns within the same Type have the same Rank). Only one pattern from each Type is set to “Active” status automatically by the Pattern Manager. That would be the pattern with the highest priority. For example, a “Running High” pattern and a “Post-Dinner High” pattern might be triggered at the same time. The “running high” may be a sign of an underlying acute abnormality, whereas the “high after dinner” may indicate a need for medication adjustment. The acute abnormality consideration would be a higher priority pattern to alert the user.

The Quarantine (Embargo) Period parameter also prevents the user from being overwhelmed with notifications. If the user was notified of a certain pattern at time instance “now”, then the Pattern Manager ensures that the user will not be notified of the same pattern again until the quarantine period expires. This means that the soonest that this particular pattern will be allowed to trigger again is at time instance “now”+the Quarantine Period. This feature also ensures that the user has time to act on improving or addressing a particular pattern before being reminded. The following table illustrates example quarantine periods:

| | | Quarantine |
|---|---|---|
| # | Pattern | Period |
| 1 | Critical Low/Meter LO | n/a |
| 2 | Critical High/Meter HI | n/a |
| 3 | Testing Frequency Low (No Goal) | 4 weeks |
| | Testing Frequency Low (Goal set) | |
| 4 | Testing Frequency Fair (No Goal Set) | 5 weeks |
| | Testing Frequency Fair (Goal Set) | |
| 5 | Testing Frequency Good (No Goal Set) | 6 weeks |
| | Testing Frequency Good (Goal Set) | |
| 6 | Testing Mostly Same Time | 3 weeks |
| 7 | High Time of Day | 4 weeks |
| 8 | Low Time of Day | 4 weeks |
| 9 | Best Time of Day | 6 weeks |
| 10 | Fasting High | 3 weeks |
| 11 | Fasting Low | 3 weeks |
| 12 | Pre-Lunch High | 4 weeks |
| 13 | Pre-Lunch Low | 4 weeks |
| 14 | Pre-Dinner High | 4 weeks |
| 15 | Pre-Dinner Low | 4 weeks |
| 16 | Post-Dinner High | 4 weeks |
| 17 | Post-Dinner Low | 3 weeks |
| 18 | Running High | 4 weeks |
| 19 | Running Low | 4 weeks |
| 20 | Day of the Week High (DOW High) | 4 weeks |
| 21 | Day of the Week Low (DOW Low) | 4 weeks |

The Incubation Period (MinReqBGhistory) parameter is related to initiation of the DMS App. To insure accuracy, the App delays responding to some triggered patterns until sufficient BG data has been received to improve reliability with respect to identifying and adjudicating patterns. Therefore, the App uses a “grace period” (MinReqBGhistory) during which triggered patterns belonging to some groups are ignored.

The Minimal Time Spacing (MinReqBGrecordTimeDiff) parameter represents the minimum allowable difference between the time stamps of two consecutive BG records in order to be included in algorithm calculations towards detecting, dismissing or improving particular IMB pattern. This feature prevents multiple readings taken around the same time to be counted as a pattern.

Patterns are characterized and presented in three different categories: Active, Additional, and Archived. An active pattern is a newly recognized pattern, either read or un-read, that is currently processing data that is considered to be the highest priority information to which the user should be alerted. The “Active” patterns are kept to a minimum at any given time to prevent the user from being overwhelmed with information. The goal of “Active” patterns is to drive a user through the specific Pattern Map Flow (user interface) at the end of which the user can reach either “Improved” or “Followed” status. Active patterns are determined by priority level (“Rank”) and “Type” consideration. Additionally, the user can select any of the Additional patterns to promote to active if the user wishes to get involved in trying to address more Pattern Map Flows. The number of Active patterns possible can be the same as the number of “Types” of patterns, for example, critical patterns (Critical High and Critical Low) as one type and three other types for patterns other than critical ones. The Active category fills before the Additional category begins to fill. The pattern detail page is accessed by selecting the pattern. An Unread pattern will prompt the user to go through the Pattern Map Flow. Selecting an Unread pattern changes its status to “Opened” (Read)′. If a Critical Pattern is detected, that pattern will be promoted to the top of the active list when triggered and only remain there until it is resolved—a non-critical in the same extreme range is taken or the sequence is “completed.” A “timed-out” timer for “Active” patterns starts when the pattern is made Active. In some embodiments, the DMS App prevents more than one pattern from the same Type to be active at the same time regardless of the total number of active patterns.

An “Additional” pattern is a recognized pattern, either read or un-read, that is posted, not collecting data and will not automatically change to “Improved” status. Additional category patterns are given more time before they time out than Active (to give the user more time to act) and move to Archived section of the Patterns Viewer. Additional category patterns are determined by priority level (Rank), Type, and the date of detection. There is no limit for the number of Additional patterns to be presented in the Patterns Viewer. To look into an Additional pattern, the user selects it and is shown the details of the pattern (top half of the pattern detail page) and the question: “Would you like to learn more about this pattern, work on improving it and move it to your Active Patterns?” Answering yes will bring them into the Pattern Map Flow for that pattern and move it to Active. Answering “No” will keep the pattern in Additional and keep the question available. To make an Additional Pattern change to Active, the user can answer the question indicating they would like to work on the pattern. By moving the Additional pattern to an Active pattern, the pattern is added to the Active section of the Patterns Viewer Screen.

The “Archived” patterns are assigned to this category by the DMS App when they reach one of the following statuses: Dismissed Reminder, Finished, Dismissed_Setup, Improved, Followed, Improved-Rework, Improved-Completed, Improved-Modified, Followed-Rework, Followed_Completed, Good_Int, Good_Note, Good_Add, or Timed-Out.

In some embodiments, the Pattern Manager may automatically transition a detected pattern to another category, state, and/or status in response to new BG records or after passage of a pre-determined amount of time. For example, if the following conditions as shown below are met, the Pattern Manager may change the category of the pattern that belongs to the Active Category to the Archived Category and assign an Improved Status:

| | | Condition to go to |
|---|---|---|
| | | Improved (Archive) from |
| # | Pattern | Active |
| 1 | Critical Low | first non-critically Low |
| | | BG reading |
| 2 | Critical High | first non-critically |
| | | High BG reading |
| 3 | Fasting High | 2 consecutive readings |
| | | below Before Meal High value |
| 4 | Fasting Low | 2 consecutive readings |
| | | above Overall Low value |
| 5 | Pre-Lunch High | 2 consecutive readings |
| | | below Before Meal High value |
| 6 | Pre-Lunch Low | 2 consecutive readings |
| | | above Overall Low value |
| 7 | Pre-Dinner High | 2 consecutive readings |
| | | below Before Meal High value |
| 8 | Pre-Dinner Low | 2 consecutive readings |
| | | above Overall Low value |
| 9 | Post-Dinner High | 2 consecutive readings |
| | | below After Meal High value |
| 10 | Post-Dinner Low | 2 consecutive readings |
| | | above Overall Low value |
| 11 | Running High | 2 consecutive readings |
| | | below After Meal High value |
| 12 | Running Low | 2 consecutive readings |
| | | above Overall Low value |
| 13 | Day of the Week High | 2 consecutive DOW |
| | (DOW High) | averages with below |
| | | After Meal High value |
| 14 | Day of the Week Low | 2 consecutive DOW |
| | (DOW Low) | averages with above |
| | | Overall Low value |

In some embodiments, if an Active pattern improves (based on receipt of new BG records) before the follow-up time, the Pattern Manager may present an “Improved Follow-up” screen and move the pattern to the Archived Category with “Improved” Status. In some embodiments, if an Active pattern improves (based on receipt of new BG records) before the follow-up time, the Pattern Manager may cancel regularly scheduled (through Pattern Map Flow) “Follow-up” notification and related reminders if they were setup during Pattern Map Flow.

In some embodiments, the Pattern Manager may change the Category of the pattern that belongs to the “Active” Category to the “Archived” Category and assign the “Timed-Out” Status if within a time interval specified in the table below, provided that none of these events has occurred:
A Pattern was not improved; or
A user did not go through the whole Pattern Map Flow to set up Follow-up; or
A Pattern has never been read.

| | | Condition for Active Pattern | |
|---|---|---|---|
| | Pattern | to go to Timed-out (Archive) | |
| 1 | Critical Low | 6 | hours |
| 2 | Critical High | 6 | hours |
| 3 | Fasting High | 10 | days |
| 4 | Fasting Low | 10 | days |
| 5 | Pre-Lunch High | 10 | days |
| 6 | Pre-Lunch Low | 10 | days |
| 7 | Pre-Dinner High | 10 | days |
| 8 | Pre-Dinner Low | 10 | days |
| 9 | Post-Dinner High | 10 | days |
| 10 | Post-Dinner Low | 10 | days |
| 11 | Running High | 10 | days |
| 12 | Running Low | 10 | days |
| 13 | Day of the Week High | 22 | days |
| | (DOW High) | | |
| 14 | Day of the Week Low | 22 | days |

(DOW Low)

In some embodiments, the Time-out period may start counting down from a Pattern Registration Moment. If an Active pattern times out (based on time), the Pattern Manager may move the pattern to the Archived Category with a Timed-out Status without notifying the user.

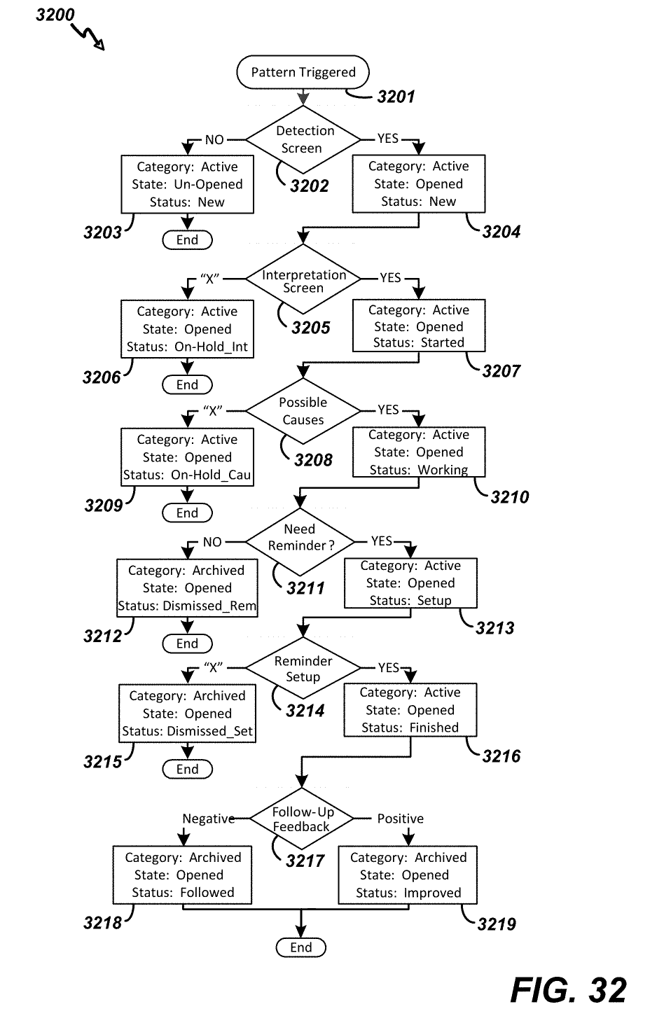

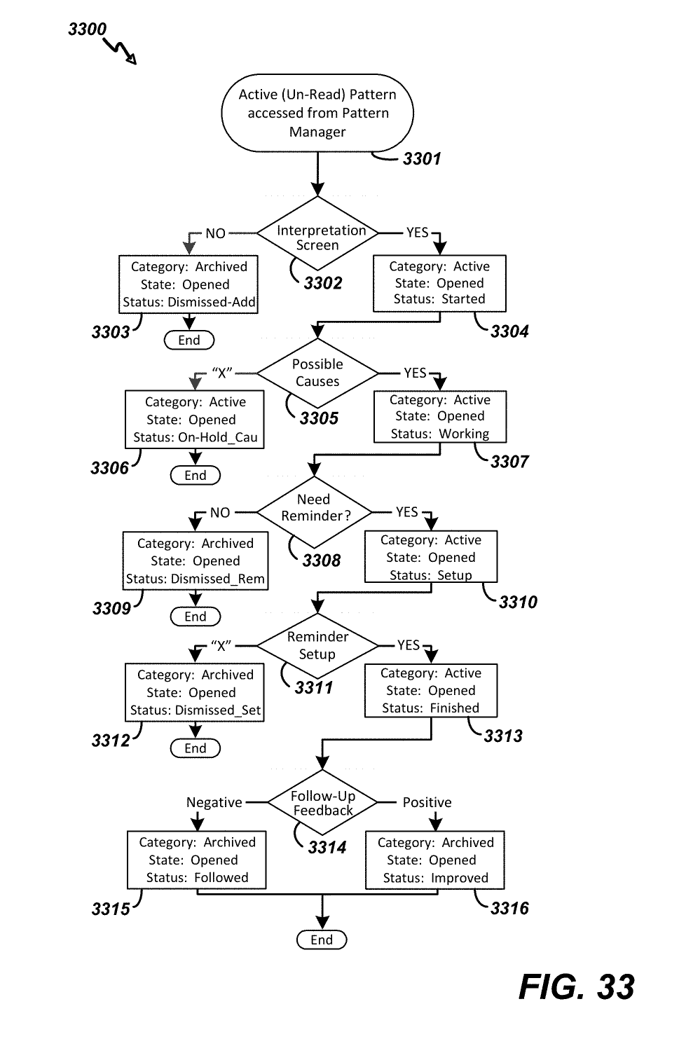

In some embodiments, transitions within Pattern Map Flow may occur. For example, during Pattern Map Flow, the Pattern Manager may assign Category, State, and/or Status according to algorithm or method 3200 for transitions within Pattern Map Flow after triggering, as illustrated in the flowchart of FIG. 32, and according to algorithm or method 3300 for transitions within Pattern Map Flow when accessed from the Pattern Manager (Active Category Un-Opened Status), as illustrated in the flowchart of FIG. 33, for the following patterns:
Fasting High
Fasting Low
Pre-Lunch High
Pre-Lunch Low
Pre-Dinner High
Pre-Dinner Low
Post Dinner High
Post Dinner Low
Recent High
Recent Low
Day of Week Low
Day of Week High

As shown in FIG. 32, in response to a triggered pattern at process block 3201, method 3200 may proceed to Detection Screen decision block 3202, wherein the outcome may result in pattern transition 3203 or 3204. From pattern transition 3204, method 3200 may proceed to Interpretation Screen decision block 3205, wherein the outcome may result in pattern transition 3206 or 3207. From pattern transition 3207, method 3200 may proceed to Possible Causes decision block 3208, wherein the outcome may result in pattern transition 3209 or 3210. From pattern transition 3210, method 3200 may proceed to Need Reminder? decision block 3211, wherein the outcome may result in pattern transition 3212 or 3213. From pattern transition 3213, method 3200 may proceed to Reminder Setup decision block 3214, wherein the outcome may result in pattern transition 3215 or 3216. From pattern transition 3216, method 3200 may proceed to Follow-Up Feedback decision block 3217, wherein the outcome may result in pattern transition 3218 or 3219.

As shown in FIG. 33, in response to an Active (Un-Read) Pattern accessed from the Pattern Manager at process block 3301, method 3300 may proceed to Interpretation Screen decision block 3302, wherein the outcome may result in pattern transition 3303 or 3304. From pattern transition 3304, method 3300 may proceed to Possible Causes decision block 3305, wherein the outcome may result in pattern transition 3306 or 3307. From pattern transition 3307, method 3300 may proceed to Need Reminder? decision block 3308, wherein the outcome may result in pattern transition 3309 or 3310. From pattern transition 3310, method 3300 may proceed to Reminder Setup decision block 3311, wherein the outcome may result in pattern transition 3312 or 3313. From pattern transition 3313, method 3300 may proceed to Follow-Up Feedback decision block 3314, wherein the outcome may result in pattern transition 3315 or 3316.

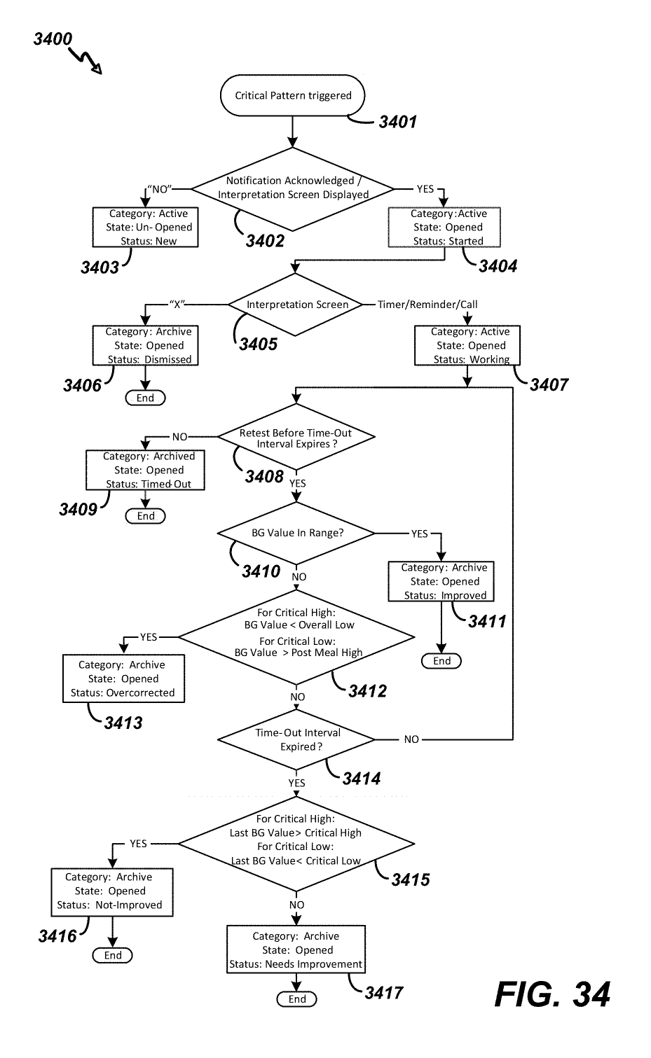

In some embodiments, other transitions within Pattern Map Flow may additionally or alternatively occur. For example, during Pattern Map Flow, the Pattern Manager may assign Category, State, and/or Status according to algorithm or method 3400, as illustrated in the flowchart of FIG. 34, for the following patterns:
Critical Low
Critical High

As shown in FIG. 34, in response to a Critical Pattern being triggered at process block 3401, method 3400 may proceed to Notification Acknowledged/Interpretation Screen Displayed decision block 3402, wherein the outcome may result in pattern transition 3403 or 3404. From pattern transition 3404, method 3400 may proceed to Interpretation Screen decision block 3405, wherein the outcome may result in pattern transition 3406 or 3407. From pattern transition 3407, method 3400 may proceed to Retest Before Time-Out Interval Expires decision block 3408, wherein a NO outcome may result in pattern transition 3409. A YES outcome at decision block 3408 may result in method 3400 proceeding to BG Value In Range? decision block 3410, wherein a YES outcome may result in pattern transition 3411 and a NO outcome may result in method 3400 proceeding to decision block 3412. At decision block 3412, the following determinations may be made: for a Critical High, is the BG Value less than Overall Low, and/or for a Critical Low, is the BG value greater than Post Meal High. If the outcome to either determination is YES, method 3400 may proceed to pattern transition 3413. If the outcome to either determination is NO, method 3400 may proceed to Time-Out Interval Expired? decision block 3414. If the outcome is NO, method 3400 may return to decision block 3408. If the outcome is YES, method 3400 may proceed to decision block 3415. At decision block 3415, the following determinations may be made: for a Critical High, is the last BG value greater than the Critical High, and/or for a Critical Low, is the last BG value less than the Critical Low. Depending on the outcome, method 3400 may proceed to pattern transition 3416 or 3417.

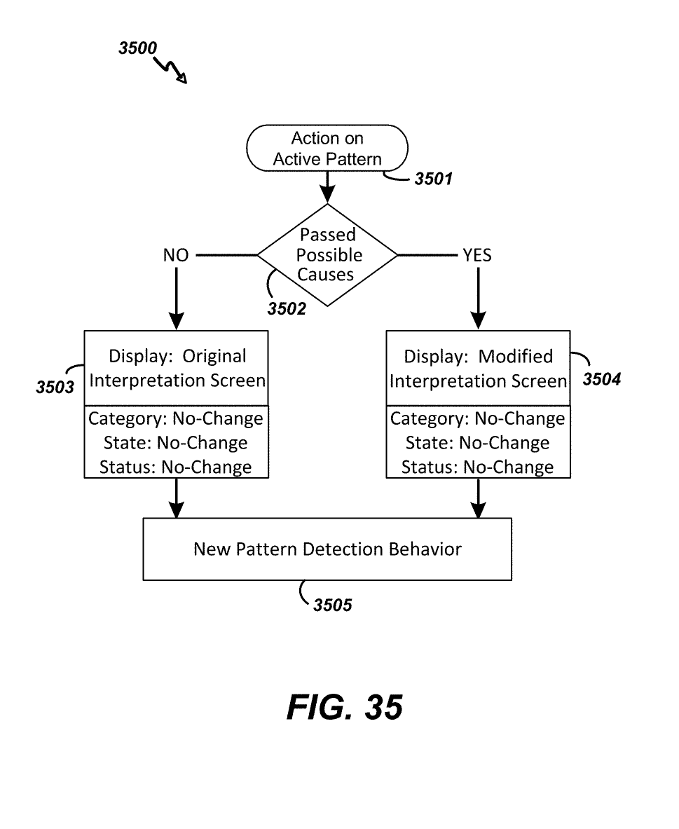

In some embodiments, transitions within Pattern Map Flow may occur because of user action within a Pattern Manager Viewer. That is, the Pattern Manager may change Category, State and/or Status of an Active pattern based on user interaction within the Pattern Manager Viewer according to, in some embodiments, algorithm or method 3500 for transitions within Pattern Map Flow after triggering, as illustrated in the flowchart of FIG. 35. When a user selects an Active pattern at process block 3501, the Pattern Manager may, via decision block 3502, display an “Original Pattern Interpretation” screen at process block 3503 or a “Modified Pattern Interpretation” screen at process block 3504.

In some embodiments, the “Modified Pattern Interpretation” screen may, in addition to the information offered in a regular “Pattern Interpretation” screen, enable a user to (1) see records that contributed to this pattern being detected (starting from the first reading contributing to the pattern being detected and ending with the reading that triggered the pattern); (2) record a Note; (3) see Related Reminders setup during the Pattern Map Flow; and/or (4) archive the pattern.

In some embodiments, when a user selects an Active pattern, and in the “Modified Pattern Interpretation” screen chooses to “Archive the Pattern,” the Pattern Manager may assign this pattern a “Dismissed” Status in the Pattern Viewer and change its actual Status to “Finished_Dismissed.” The Pattern Manager may, in some embodiments, allow an “Archived” pattern to change its Status from Unopened to Opened (only in Pattern Manager but not in Pattern Viewer). In some embodiments, the Pattern Manager may not allow an “Archived” pattern to change Category and State.

In some embodiments, when a user selects an Archived pattern that is in either “Dismissed” (“Dismissed_Rem”, “Dismissed_Setup”) or “Timed-Out” status, the Pattern Manager may display a “Modified Pattern Interpretation” screen at process block 3504. The “Modified Pattern Interpretation” screen may, in addition to the information offered in a regular “Pattern Interpretation” screen, enable a user to (1) see the state of the pattern, and (2) select from the following options: (a) see records that contributed to this pattern being detected (starting from the first reading contributing to the pattern being detected and ending with the last reading related to the pattern before the pattern was dismissed or timed out), and (b) record a Note.

In some embodiments, when a user selects an Archived pattern that is either in “Improved” or “Followed” status, the Pattern Manager may display a “Modified Pattern Interpretation” screen that may in addition to the information offered in a regular “Pattern Interpretation” screen, enable a user to (1) see the status, and (2) select from the following options: (a) see records that contributed to this pattern being detected and Improved or Not Improved (Followed), meaning all records related to this particular pattern starting with the first reading contributing to the pattern being detected and ending with the last reading related to the pattern before a Follow up interval expires; (b) record a Note; and (c) see Related Reminders setup during the Pattern Map Flow.

In some embodiments, when a user selects an Archived pattern that belongs to the group of Critical Patterns, the Pattern Manager may display a “Critical Pattern Follow-Up” screen that may in addition to the information offered in a regular “Pattern Interpretation” screen, enable users to (1) see a detailed status explanation; (2) record a Note; and (3) see Related Readings setup during the Pattern Map Flow (e.g., all readings starting with the one that triggered the pattern and ending with the last reading related to the pattern recorded before Pattern Improvement moment or Pattern Time Out interval expiration).

In some embodiments, critical patterns may take precedence over non-critical patterns, and the Pattern Manager may store and not allow manual deletion of any Active or Archived Patterns. In some embodiments, a maximum of the 50 most current Active and Archived Patterns (i.e., first-in-first-out) may be stored and retained for up to 90 days, wherein patterns older than 90 days may be deleted.

Returning to FIG. 35, method 3500 may proceed from either process block 3503 or 3504 to New Pattern Detection Behavior process block 3505, where new patterns may be detected by the Pattern Manager as described above.

Numerous embodiments are described in this disclosure, and are presented for illustrative purposes only. The described embodiments are not, and are not intended to be, limiting in any sense. The presently disclosed inventions are widely applicable to numerous embodiments, as is readily apparent from the disclosure. One of ordinary skill in the art will recognize that the disclosed inventions may be practiced with various modifications and alterations, such as structural, logical, software, and electrical modifications. Although particular features of the disclosed inventions may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments nor a listing of features of the invention that must be present in all embodiments.

The Title (set forth at the beginning of the first page of this disclosure) is not to be taken as limiting in any way as the scope of the disclosed inventions.

The term “product” means any machine, manufacture and/or composition of matter as contemplated by 35 U.S.C. § 101, unless expressly specified otherwise.

Each process (whether called a method, class behavior, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device, component, structure, or article is described herein, more than one device, component, structure or article (whether or not they cooperate) may alternatively be used in place of the single device, component or article that is described. Accordingly, the functionality that is described as being possessed by a device may alternatively be possessed by more than one device, component or article (whether or not they cooperate).

Similarly, where more than one device, component, structure, or article is described herein (whether or not they cooperate), a single device, component, structure, or article may alternatively be used in place of the more than one device, component, structure, or article that is described. For example, a plurality of computer-based devices may be substituted with a single computer-based device. Accordingly, the various functionality that is described as being possessed by more than one device, component, structure, or article may alternatively be possessed by a single device, component, structure, or article.

The functionality and/or the features of a single device that is described may be alternatively embodied by one or more other devices that are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this disclosure are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining, recognizing, and the like.

A “display” as that term is used herein is an area that conveys information to a viewer. The information may be dynamic, in which case, an LCD, LED, CRT, Digital Light Processing (DLP), rear projection, front projection, or the like may be used to form the display.

The present disclosure may refer to a “control system”, application, or program. A control system, application, or program, as that term is used herein, may be a computer processor coupled with an operating system, device drivers, and appropriate programs (collectively “software”) with instructions to provide the functionality described for the control system. The software is stored in an associated memory device (sometimes referred to as a computer readable medium). While it is contemplated that an appropriately programmed general purpose computer or computing device may be used, it is also contemplated that hard-wired circuitry or custom hardware (e.g., an application specific integrated circuit (ASIC)) may be used in place of, or in combination with, software instructions for implementation of the processes of various embodiments. Thus, embodiments are not limited to any specific combination of hardware and software.

A “processor” means any one or more microprocessors, Central Processing Unit (CPU) devices, computing devices, microcontrollers, digital signal processors, or like devices. Exemplary processors are the INTEL PENTIUM or AMD ATHLON processors.

The term “computer-readable medium” refers to any statutory medium that participates in providing data (e.g., instructions) that may be read by a computer, a processor or a like device. Such a medium may take many forms, including but not limited to non-volatile media, volatile media, and specific statutory types of transmission media. Non-volatile media include, for example, optical or magnetic disks and other persistent memory. Volatile media include DRAM, which typically constitutes the main memory. Statutory types of transmission media include coaxial cables, copper wire and fiber optics, including the wires that comprise a system bus coupled to the processor. Common forms of computer-readable media include, for example, a floppy disk, a flexible disk, hard disk, magnetic tape, any other magnetic medium, a CD-ROM, Digital Video Disc (DVD), any other optical medium, punch cards, paper tape, any other physical medium with patterns of holes, a RAM, a PROM, an EPROM, a FLASH-EEPROM, a USB memory stick, a dongle, any other memory chip or cartridge, a carrier wave, or any other medium from which a computer can read. The terms “computer-readable memory” and/or “tangible media” specifically exclude signals, waves, and wave forms or other intangible or non-transitory media that may nevertheless be readable by a computer.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols. For a more exhaustive list of protocols, the term “network” is defined below and includes many exemplary protocols that are also applicable here.

It will be readily apparent that the various methods and algorithms described herein may be implemented by a control system and/or the instructions of the software may be designed to carry out the processes of the present invention.

Where databases and/or data structures are described, it will be understood by one of ordinary skill in the art that (i) alternative database structures to those described may be readily employed, and (ii) other memory structures besides databases may be readily employed. Any illustrations or descriptions of any sample databases/data structure presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models, hierarchical electronic file structures, and/or distributed databases) could be used to store and manipulate the data types described herein. Likewise, object methods or behaviors of a database can be used to implement various processes, such as those described herein. In addition, the databases may, in a known manner, be stored locally or remotely from a device that accesses data in such a database. Furthermore, while unified databases may be contemplated, it is also possible that the databases may be distributed and/or duplicated amongst a variety of devices.

As used herein a “network” generally refers to an information or computing network that can be used to provide an environment wherein one or more computing devices may communicate with one another. Such devices may communicate directly or indirectly, via a wired or wireless medium such as the Internet, LAN, WAN or Ethernet (or IEEE 802.3), Token Ring, or via any appropriate communications means or combination of communications means. Exemplary protocols include but are not limited to: Bluetooth™, Time Division Multiple Access (TDMA), Code Division Multiple Access (CDMA), Global System for Mobile communications (GSM), Enhanced Data rates for GSM Evolution (EDGE), General Packet Radio Service (GPRS), Wideband CDMA (WCDMA), Advanced Mobile Phone System (AMPS), Digital AMPS (D-AMPS), IEEE 802.11 (WI-FI), IEEE 802.3, SAP, the best of breed (BOB), system to system (S2S), or the like. Note that if video signals or large files are being sent over the network, a broadband network may be used to alleviate delays associated with the transfer of such large files, however, such is not strictly required. Each of the devices is adapted to communicate on such a communication means. Any number and type of machines may be in communication via the network. Where the network is the Internet, communications over the Internet may be through a website maintained by a computer on a remote server or over an online data network including commercial online service providers, bulletin board systems, and the like.

In yet other embodiments, the devices may communicate with one another over RF, cable TV, satellite links, and the like. Where appropriate encryption or other security measures such as logins and passwords may be provided to protect proprietary or confidential information.

Communication among computers and devices may be encrypted to insure privacy and prevent fraud in any of a variety of ways well known in the art. Appropriate cryptographic protocols for bolstering system security are described in Schneier, APPLIED CRYPTOGRAPHY, PROTOCOLS, ALGORITHMS, AND SOURCE CODE IN C, John Wiley & Sons, Inc. 2d ed., 1996, which is incorporated by reference in its entirety.

It will be readily apparent that the various methods and algorithms described herein may be implemented by, e.g., appropriately programmed general purpose computers and computing devices. Typically a processor (e.g., one or more microprocessors) will receive instructions from a memory or like device, and execute those instructions, thereby performing one or more processes defined by those instructions. Further, programs that implement such methods and algorithms may be stored and transmitted using a variety of media (e.g., computer readable media) in a number of manners. In some embodiments, hard-wired circuitry or custom hardware may be used in place of, or in combination with, software instructions for implementation of the processes of various embodiments. Thus, embodiments are not limited to any specific combination of hardware and software. Accordingly, a description of a process likewise describes at least one apparatus for performing the process, and likewise describes at least one computer-readable medium and/or memory for performing the process. The apparatus that performs the process can include components and devices (e.g., a processor, input and output devices) appropriate to perform the process. A computer-readable medium can store program elements appropriate to perform the method.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

The foregoing description discloses only exemplary embodiments of the invention. Modifications of the above disclosed apparatus and methods which fall within the scope of the invention will be readily apparent to those of ordinary skill in the art. For example, although the examples discussed above are illustrated for an medical device market, embodiments of the invention can be implemented for other markets.

Accordingly, while the present invention has been disclosed in connection with exemplary embodiments thereof, it should be understood that other embodiments may fall within the spirit and scope of the invention, as defined by the following claims.

What is claimed is:

1. Apparatus for diabetes management, the apparatus comprising:
a portable diabetes management system (DMS) device including a processor, a data storage device, a touchscreen display, wireless communications facilities, a pattern recognition engine stored in the data storage device and executable in the processor, and a user interface structure stored in the data storage device and executable in the processor, the user interface structure comprising a plurality of user interface displays configured to be displayed on the touchscreen display; wherein:
one of the plurality of user interface displays includes a listing of a selectable subset of a plurality of different patterns based on blood glucose measurement data received by the DMS device, the selectable subset of patterns based upon a frequency with which the different patterns are detected by the pattern recognition engine.

2. The apparatus of claim 1, wherein the different patterns include at least one of a critical low meter reading, critical high meter reading, test frequency low, test frequency fair, test frequency good, testing mostly same time, high for the time of day, low for the time of day, best time of day, fasting high, fasting low, pre-lunch high, pre-lunch low, pre-throughout high, pre-dinner low, post dinner high, post dinner low, running high, running low, day of week low, and day of week high.

3. The apparatus of claim 1, wherein the pattern recognition engine comprises a plurality of algorithms, each algorithm configured to identify a respective one of the different patterns.

4. The apparatus of claim 1, wherein the pattern recognition engine is configured to recognize patterns based on 14-21 days of the blood glucose measurement data received by the DMS device.

5. The apparatus of claim 1, wherein each user interface display is linked to or reachable via at least one other of the plurality of user interface displays or is presented as a result of a pattern being detected by the pattern recognition engine.

6. The apparatus of claim 1, wherein one of the plurality of user interface displays also includes a screen for selecting a number of blood glucose tests to be performed per week.

7. The apparatus of claim 6, wherein one of the plurality of user interface displays also includes a reminder screen to perform a blood glucose test, the reminder screen to be displayed on the touchscreen display in response to the pattern recognition engine detecting less than the number of blood glucose tests to be performed per week selected via the screen for selecting a number of blood glucose tests to be performed per week.

8. The apparatus of claim 1, wherein one of the plurality of user interface displays also includes a pattern manager screen that includes interactive lists of active, additional, and archived detected patterns.

9. The apparatus of claim 1, wherein one of the plurality of user interface displays also includes a pattern detail screen that includes a summary area, a graph area, a status area, an explanation area, and a “further links” area.

10. A method for diabetes management, the method comprising:
receiving blood glucose measurements at a portable wireless device from a blood glucose meter;
storing the blood glucose measurements in a data storage device of the portable wireless device;
recognizing one or more patterns with a processor of the portable wireless device based on the blood glucose measurements, the processor executing a pattern recognition engine stored in the data storage device; and
prompting a user via a user interface of the portable wireless device to take an action in response to the recognizing one or more of the patterns.

11. The method of claim 10, wherein the patterns include at least one of a critical low meter reading, critical high meter reading, test frequency low, test frequency fair, test frequency good, testing mostly same time, high for the time of day, low for the time of day, best time of day, fasting high, fasting low, pre-lunch high, pre-lunch low, pre-throughout high, pre-dinner low, post dinner high, post dinner low, running high, running low, day of week low, and day of week high.

12. The method of claim 10, wherein the recognizing one or more of the patterns is based on 14-21 days of the receiving blood glucose measurements at the portable wireless device.

13. The method of claim 10, further comprising storing a user interface structure in the data storage device, the user interface structure executable in the processor.

14. The method of claim 10, wherein the prompting the user to take an action comprises prompting the user to set up a pattern goal related to the one or more patterns recognized.

15. The method of claim 10, wherein the prompting the user to take an action comprises at least one of prompting the user to test their blood glucose level, to take their medication, to log their activity, and to log their carbohydrate intake.

16. The method of claim 10, wherein the prompting the user via the user interface to take an action comprises generating and presenting on a display of the portable wireless device at least one of a recommendation, a reminder, and a warning to the user.

17. The method of claim 16, further comprising limiting the frequency with which the reminder is presented.

18. The method of claim 16, further comprising prioritizing the presenting on the display the at least one of the recommendation, the reminder, and the warning based on pre-defined priorities stored in the data storage device.

19. The method of claim 10, wherein the prompting the user via the user interface to take an action comprises presenting on a display of the portable wireless device at least one of a recommendation, a reminder, and a warning with higher intensity or more frequently than the others of the recommendation, the reminder, and the warning.

20. The method of claim 19, wherein the higher intensity includes one of larger text, brighter highlighting, different colors, and sound.
